(19) United States
(12) Patent Application Publication
(10) Pub. No.: US 2025/0009916 A1
(43) Pub. Date: Jan. 9, 2025

(54) COMBINATION THERAPY FOR TREATING LUNG CANCER

(71) Applicants: Novartis AG, Basel (CH); Advanced Accelerator Applications, Rueil-Malmaison (FR)

(72) Inventors: Paola Daniela AIMONE, Rixheim (FR); Daniela CHICCO, Bollengo (IT); Ilia FOLITAR, Riehen (CH); Maurizio MARIANI, Ivrea (IT)

(21) Appl. No.: 18/708,825

(22) PCT Filed: Nov. 10, 2022
(86) PCT No.: PCT/IB2022/060840

§ 371 Date: May 9, 2024

Related U.S. Application Data
(60) Provisional application No. 63/278,621, filed on Nov. 12, 2021.

(51) Int. Cl. A61K 51/08; A61K 31/282; A61K 31/7048; A61K 39/395; A61K 45/06; A61P 35/00

(52) U.S. Cl. CPC A61K 51/088; A61K 31/282; A61K 31/7048; A61K 39/3955; A61K 45/06; A61K 51/083; A61P 35/00; A61K 2121/00

(57) ABSTRACT

The present invention relates to methods for treating small cell lung cancer (SCLC), in particular small cell lung cancer (SCLC) in a subject in need thereof wherein a therapeutically efficient amount of a radiopharmaceutical compound comprising a SSTR binding moiety, in particular [“Lu] Lu-DOTATE is administered to said subject in combination with one or more chemotherapeutic agents, such as carboplatin and etoposide, and, optionally an immune-oncology (I/O) agent, such as tislelizumab.

### FIELD OF THE INVENTION

The present invention relates to methods for treating small cell lung cancer (SCLC) in a subject in need thereof wherein a therapeutically efficient amount of a radiopharmaceutical compound comprising a SSTR binding moiety is administered to said subject in combination with a chemotherapeutic agent and, optionally, an immune-oncology (I/O) agent.

### SEQUENCE LISTING

This application contains a sequence listing that has been submitted electronically in XML format and is hereby incorporated by reference in its entirety. The XML copy, created on Nov. 7, 2022, is named PAT059204_SL.xml.

### BACKGROUND

Lung cancer is the most common cause of cancer death accounting for 1.80 million deaths worldwide in 2020 (WHO Cancer fact sheet 2021), while the cases of lung cancer diagnosis and deaths continue to rise over years (Bade BC, Dela Cruz CS Lung Cancer 2020: Clin Chest Med; 41:1-24). Small cell lung carcinoma (SCLC) is the most lethal and aggressive subtype of lung cancer representing around 10-15% of all lung cancers with an estimated median survival of 18-23 months for Limited Stage SCLC (LS-SCLC) patients and 8-10 months for Extensive Stage SCLC (ES-SCLC) patients (Dela Cruz CS, Tanoue LT, Matthay RA (2011) Clin Chest Med; 32:605-44, Dayen C, Debieuvre D, Molinier O, et al (2017) New insights into stage and prognosis in small cell lung cancer: an analysis of 968 cases. J Thorac Dis; 9 (12): 5101-11).

Somatostatin receptors (SSTR) are expressed in 30% to 50% of patients with SCLC as evidenced by immunohistochemistry and [68Ga] Ga-DOTA-TATE PET/CT scan (Lapa C, Hänscheid H, Wild V, et al (2016) Oncotarget; 7 (15): 20033-40, Lehman JM, Hoeksema MD, Staub J, et al (2019) Int J Cancer; 144:1104-14). It was shown that somatostatin receptor 2 signaling promotes growth and survival in SCLC, and a high SSTR-2 expression is correlated with worse patient survival (Lehman JM, Hoeksema MD, Staub J, et al (2019) Int J Cancer; 144:1104-14).

SCLC is known to be a radiosensitive tumor suitable for external thoracic radiotherapy which is mostly used as concurrent chemoradiotherapy in patients with LS-SCLC. In ES-SCLC radiotherapy can be used for symptom control.

The current standard first-line treatment for patients with ES-SCLC is systemic platinum based chemotherapy (cisplatin or carboplatin, with etoposide, a topoisomerase II inhibitor) in combination with immune checkpoint inhibitors (atezolizumab or durvalumab) (NCCN Guidelines Small Cell Lung Cancer Version 3.2021; Dingemans AMC, Fruh M, Ardizzoni A, et al (2021) 32 (7): 839-53).

In clinical setting, the hypothesis that immune checkpoint inhibitors would act synergistically in combination with [¹⁷⁷Lu] Lu-DOTA-TATE was evaluated in a phase I study in 9 patients with either progressive refractory ES-SCLC, or non-progressing ES-SCLC after first line platinum-based chemotherapy, or advanced grade I-II pulmonary NETs (Kim C, Liu SV, Subramaniam DS, et al (2020) J Immunother Cancer; 8: e000980).

Similarly to the synergistic effect of the combination of PRRT with I/O for certain cancers (also reported in WO2016/207732 and WO2020021465), the combination of PRRT with carboplatin/etoposide was evaluated and showed a significantly prolonged survival versus PRRT or chemotherapy alone in preclinical setting (Lewin J, Cullinane C, Akhurst T, et al (2015) Eur J Nucl Med Mol Imaging; 42:25-32). These preclinical findings have been translated into clinical use in a patient with metastatic relapsed small cell cancer of extrapulmonary origin with high SSTR2 expression, who received PRRT plus etoposide (carboplatin was avoided due to renal function). The patient experienced a complete metabolic response for 4 months and clinical improvement. The treatment was well tolerated apart from mild fatigue, nausea, grade 2 anemia and grade 3 thrombocytopenia. The patient experienced disease progression after 4.5 months of treatment start (Lewin J, Cullinane C, Akhurst T, et al (2015) Eur J Nucl Med Mol Imaging; 42:25-32).

Accordingly, despite some progress, there are still limited therapeutic options for the treatment of SCLC, in particular ES-SCLC, and the overall prognosis of patients with ES-SCLC remains very poor.

The present disclosure provides novel therapeutic options for treating SCLC, in particular ES-SCLC, in particular based on the combination of an SSTR peptide targeted radionuclide therapy (e.g. ¹⁷⁷Lu-DOTA-TATE) with chemotheray (e.g carboplatine and etoposide) and immune checkpoint inhibitor (e.g. tislelizumab) in patients with SSTR-positive SCLC, in particular ES-SCLC.

### LEGENDS OF THE FIGURES

FIG. 1: Clinical study design

### SUMMARY

### Specific Embodiments

1. A radiopharmaceutical compound for use in treating small cell lung cancer (SCLC), in particular extensive stage small cell lung cancer (ES-SCLC), in a human subject in need thereof, wherein a therapeutically efficient amount of radiopharmaceutical compound comprising a somatostatin receptor binding molecule is administered to said subject, in combination, preferably concomitantly, with a therapeutically efficient amount of one or more chemotherapeutic agents.
2. The radiopharmaceutical compound for use of embodiment 1, wherein said radiopharmaceutical compound is a compound of formula:
   M-C—S—P wherein:
   M is a radionuclide;
   C is a chelating agent capable of chelating said radionuclide;
   S is an optional spacer covalently linked between C and P;
   P is a somatostatin receptor binding peptide covalently linked to C, either directly or indirectly via S.
3. The radiopharmaceutical compound for use of embodiment 2, wherein M is selected from ⁹⁰Y, ¹³¹I, ¹²¹Sn, ¹⁸⁶Re, ¹⁸⁸Re, ⁶⁴Cu, ⁶⁷Cu, ⁵⁹Fe, ⁸⁹Sr, ¹⁹⁸Au, ²⁰³Hg, ²¹²Pb, ¹⁶⁵Dy, ¹⁰³Ru, ¹⁴⁹Tb, ¹⁶¹Tb, ²¹³Bi, ¹⁶⁶Ho, ¹⁶⁵Er, ¹⁶⁹Er, ¹⁵³Sm, ¹⁷⁷Lu, ²¹³Bi, ²²³Ra, ²²⁵ Ac, ²²⁷Ac, ²²⁷Th, ²¹¹At, ⁶⁷Cu, ¹⁸⁶Re, ¹⁸⁸Re, ¹⁶¹Tb, ¹⁷⁵Yb, ¹⁰⁵Rh, ¹⁶⁶ Dy, ¹⁹⁹Au, ⁴⁴Sc, ¹⁴⁹Pm, ¹⁵¹Pm, ¹⁴²Pr, ¹⁴³Pr, ⁷⁶As, ¹¹¹Ag and ⁴⁷Sc, preferably is ¹⁷⁷Lu.
4. The radiopharmaceutical compound for use according to embodiment 2 or 3, wherein C is selected from DOTA (tetraxetan), trizoxetan, DOTAGA, DTPA, NTA, EDTA, DO3A, TETA, NOTA, NOTAGA, NODAGA, NODAPA, and AAZTA (e.g. AAZTA5) chelating agent, preferably is DOTA, DOTAGA, NOTA or DTPA chelating agent, and more preferably is DOTA chelating agent.
5. The radiopharmaceutical compound for use according to any of the embodiments 1-4, wherein P is selected from octreotide, octreotate, satoreotide, lanreotide, vapreotide, and pasireotide, preferably selected from octreotide and octreotate.
6. The radiopharmaceutical compound for use according to any of the embodiments 1-5, wherein the radiopharmaceutical compound is selected from DOTA-OC, DOTA-TOC (edotreotide), DOTA-NOC, DOTA-TATE (oxodotreotide), satoreotide tetraxetan, DOTA-LAN, and DOTA-VAP, preferably selected from DOTA-TOC and DOTA-TATE, more preferably is DOTA-TATE.
7. The radiopharmaceutical compound for use according to any of the embodiments 1-6, wherein the radiopharmaceutical compound is [¹⁷⁷Lu] Lu-DOTA-TOC (¹⁷⁷Lu-edotreotide) or [¹⁷⁷Lu] Lu-DOTA-TATE (¹⁷⁷Lu-oxodotreotide), more preferably [¹⁷⁷Lu] Lu-DOTA-TATE (¹⁷⁷Lu-oxodotreotide).
8. The radiopharmaceutical compound for use according to any of the embodiments 1-7, wherein said subject has not received prior systematic treatment for SCLC, in particular ES-SCLC, in particular said subject has not received prior chemotherapy for treating SCLC, in particular ES-SCLC.
9. The radiopharmaceutical compound for use according to any of the embodiments 1-8, wherein said subject is newly diagnosed with SCLC, in particular ES-SCLC.
10. The radiopharmaceutical compound for according to any of the embodiments 1-9, wherein said subject has been selected for the treatment by SPECT/CT or PET/CT or SPECT/MRI, PET/MRI imaging with the same organic compound as used for the radiopharmaceutical treatment but with a radiometal suitable for imaging, preferably 68Ga, 67Ga or 64Cu, more preferably 68Ga.
11. The radiopharmaceutical compound for use according to any of the embodiments 1-10, wherein said subject has been diagnosed as SSTR positive by imaging positron emission tomography (PET) scan in at least one target or non-target lesion, preferably with [68Ga] Ga-DOTA-TATE imaging PET scan.
12. The radiopharmaceutical compound for use according to any of the embodiments 1-11, wherein said one or more chemotherapeutic agents include carboplatin (preferably carboplatin AUC 5 on day 1 of every 3-week-cycle) and etoposide (preferably administered at a daily dose of 100 mg/m2 on days 1, 2 and 3 of every 3-week-cycle).
13. The radiopharmaceutical compound for use according to any of the embodiments 1-12, wherein said radiopharmaceutical compound is administered 1 to 8 times, preferably 2 to 7 times, for example 4 to 6 times, wherein there is a treatment interval between every two administrations of said radiopharmaceutical compound.
14. The radiopharmaceutical compound for use according to any of the embodiments 1-13, wherein each administration of said radiopharmaceutical compound comprises a treatment interval of 2 weeks, or 3 weeks, or 4 weeks, or 5 weeks or even 6 weeks, preferably 3 and/or 6 weeks.
15. The radiopharmaceutical compound for use according to any of the embodiments 1-14, wherein said one or more chemotherapeutic agents is (are) administered in combination, preferably concomitantly during an induction period comprising two administrations of said radiopharmaceutical compound, preferably a first administration of said radiopharmaceutical compound at week 1 of the first administration of a chemotherapeutic agent, for example on either Day 3, 4 or 5 of Week 1, and a second administration of said radiopharmaceutical compound between Week 6 and Week 8, preferably Week 7.
16. The radiopharmaceutical compound for use according to embodiment 15, further comprising a maintenance period following the induction period, comprising 1 to 4 administrations of said radiopharmaceutical compound, preferably every 3 weeks.
17. The radiopharmaceutical compound for use according to any of embodiments 1-16, comprising in combination, preferably concomitantly administering a therapeutically efficient amount of one or two more immune-oncology (I-O) therapeutic agents, preferably selected from the group consisting of PD-1 inhibitors, PD-L1 inhibitors, or CTLA4 inhibitors, LAG-3 inhibitors, TIM-3 inhibitors, TIGIT inhibitors, GITR antagonists, TGF-b inhibitors, IL15/IL15RA complexes, CD40/CD40L complexes, OX40 inhibitors, 4-1BB/CD137 complexes, ICOS inhibitors, CD47 inhibitors, VISTA inhibitors, GD-2 inhibitors, and B7/H3 inhibitors, cytokines (e.g., interferon, interlukin), cellular immunotherapies, and cancer vaccines, more preferably are PD-1 inhibitors, PD-L1 inhibitors, or CTLA4 inhibitors, or a combination thereof. In some embodiments, the inhibitors used herein are antibodies.
18. The radiopharmaceutical compound for use according to embodiment 17, wherein
   (i) said one or more chemotherapeutic agents is (are) administered in combination, preferably concomitantly during an induction period comprising two administrations of said radiopharmaceutical compound, preferably a first administration of said radiopharmaceutical compound at Week 1 of the first administration of a chemotherapeutic agent, for example on either Day 3, 4 or 5 of Week 1, and a second administration of said radiopharmaceutical compound between Week 6 and Week 8, preferably Week 7, and
   (ii) said immune-oncology agent is administered in combination, preferably concomitantly with said chemotherapeutic agent on Week 1, preferably the day of first administration of a chemotherapeutic agent and every 3 weeks during the induction period.
19. The radiopharmaceutical compound for use according to embodiment 18, further comprises a maintenance period following the induction period comprising
   (i) 1 to 4 administrations of said radiopharmaceutical compound every 3 weeks and
   (ii) 1 to 4 administration of said immune-oncology agent every 3 weeks.
20. The radiopharmaceutical compound for use according to any of embodiments 17 to 19, wherein said PD-1, PD-L1 or CTLA-4 inhibitors are selected from the group consisting of anti-PD1, anti-PD-L1 or anti-CTLA-4 antibodies, for example selected from the group consisting of nivolumab (Bristol-Myers Squibb), ipilimumab, PDR001/spartalizumab (Novartis), Keytruda/pembrolizumab/MK-3475/lambrolizumab (Merk & Co), pidilizumab, durvalumab/MEDI4736, atezolizumab/MPDL3280A/Tecentriq/RG7446 (Roche), avelumab, MEDI0680 (AMP-514, Medimmune), REGN2810/Cemiplimab (Regeneron), TSR-042/Dostarlimab/Dostarlimab-gxly (Tesaro), PF-06801591/Sananlimab (Pfizer), BGB-A317/tislelizumab (Beigene), BGB-108, INCSHR1210/Camrelizumab (Incyte), and AMP-224 (Amplimmune).
21. The radiopharmaceutical compound for use according to embodiment 20, wherein said PD-1, PD-L1 or CTLA-4 inhibitor is tislelizumab, and is preferably administered at a dose of from about 200 mg to about 500 mg, more preferably from about 200 mg to about 400 mg, even more preferably either from about 200 mg to about 300 mg, even more preferably either about 200 mg or about 300 mg, even more preferably 200 mg.
22. The radiopharmaceutical compound for use according to any of embodiments 1-21 wherein said subject has not received prior systematic treatment for SCLC, in particular ES-SCLC, in particular said subject has not received prior chemotherapy for treating SCLC, in particular ES-SCLC, wherein said radiopharmaceutic compound is [¹⁷⁷Lu] Lu-DOTA-TATE, said one or more chemotherapeutic agent is carboplatin and etoposide, and said use further comprises administering a therapeutically efficient amount of tislelizumab in combination, preferably concomitantly with [177LU] Lu-DOTA-TATE administration.
23. The radiopharmaceutical compound for use according to embodiment 22, wherein
   (i) said carboplatin and etoposide are administered in combination, preferably concomitantly during an induction period comprising two administrations of [177LU] Lu-DOTA-TATE, preferably a first administration of [177LU] Lu-DOTA-TATE at Week 1 after the first administration of carboplatin and/or etoposide, for example on either Day 3, 4 or 5 of Week 1, and a second administration of [177LU] Lu-DOTA-TATE between Week 6 and Week 8, preferably Week 7, and
   (ii) tislelizumab is administered in combination, preferably concomitantly with carboplatin and etoposide on Week 1, preferably the day of first administration of carboplatin and every 3 weeks during the induction period.
24. The radiopharmaceutical compound for use according to embodiment 23, wherein said use further comprises a maintenance period, following the induction period, comprising
   (i) 1 to 4 administrations of [177LU] Lu-DOTA-TATE every 3 weeks and
   (ii) 1 to 4 administrations of tislelizumab agent every 3 weeks.
25. The radiopharmaceutical compound for use of embodiments 1-24, wherein said radiopharmaceutical compound is administered at a dose (i.e. daily dose, dose for each administration, non-cumulative dose) ranging between 0.925 GBq (25 mCi) to 29.6 GBq (800 mCi), preferably between 1.48 GBq (40 mCi) to 18.5 GBq (500 mCi), preferably between 1.85 GBq (50 mCi) to 14.8 GBq (400 mCi), more preferably between 3.7 GBq (100 mCi) to 11.1 GBq (300 mCi), even more preferably of around 3.7 GBq (100 mCi), 5.55 GBq (150 mCi), 7.4 GBq (200 mCi) or 9.25 GBq (250 mCi).
26. A method for treating small cell lung cancer (SCLC), in particular extensive stage small cell lung cancer (ES-SCLC), in a human subject in need thereof, said method comprising administering to said subject, a therapeutically efficient amount of a radiopharmaceutical compound comprising a somatostatin receptor binding molecule, in combination, preferably concomitantly, with a therapeutically efficient amount of one or more chemotherapeutic agents.
27. The method of embodiment 26, wherein said radiopharmaceutical compound is a compound of formula:
   M-C—S—P wherein:
   M is a radionuclide;
   C is a chelating agent capable of chelating said radionuclide;
   S is an optional spacer covalently linked between C and P;
   P is a somatostatin receptor binding peptide covalently linked to C, either directly or indirectly via S.
28. The method of embodiment 27, wherein M is selected from ⁹⁰Y, ¹³¹I, ¹²¹Sn, ¹⁸⁶Re, ¹⁸⁸Re, ⁶⁴Cu, ⁶⁷Cu, ⁵⁹Fe, ⁸⁹Sr, ¹⁹⁸Au, ²⁰³Hg, ²¹²Pb, ¹⁶⁵Dy, ¹⁰³Ru, ¹⁴⁹Tb, ¹⁶¹Tb, ²¹³Bi, ¹⁶⁶Ho, ¹⁶⁵Er, ¹⁶⁹Er, ¹⁵³Sm, ¹⁷⁷Lu, ²¹³Bi, ²²³Ra, ²²⁵ Ac, ²²⁷Ac, ²²⁷Th, ²¹¹At, ⁶⁷Cu, ¹⁸⁶Re, ¹⁸⁸Re, ¹⁶¹Tb, ¹⁷⁵Yb, ¹⁰⁵Rh, ¹⁶⁶Dy, ¹⁹⁹Au, ⁴⁴Sc, ¹⁴⁹Pm, ¹⁵¹Pm, ¹⁴²Pr, ¹⁴³Pr, ⁷⁶As, ¹¹¹Ag and ⁴⁷Sc, preferably is ¹⁷⁷Lu.
29. The method of embodiment 27 or 28, wherein C is selected from DOTA (tetraxetan), trizoxetan, DOTAGA, DTPA, NTA, EDTA, DO3A, TETA, NOTA, NOTAGA, NODAGA, NODAPA, and AAZTA (e.g. AAZTA5) chelating agent, preferably is DOTA, DOTAGA, NOTA or DTPA chelating agent, and more preferably is DOTA chelating agent.
30. The method of any of the embodiments 26-29, wherein P is selected from octreotide, octreotate, satoreotide, lanreotide, vapreotide, and pasireotide, preferably selected from octreotide and octreotate.
31. The method any of the embodiments 26-30, wherein the radiopharmaceutical compound is selected from DOTA-OC, DOTA-TOC (edotreotide), DOTA-NOC, DOTA-TATE (oxodotreotide), satoreotide tetraxetan, DOTA-LAN, and DOTA-VAP, preferably selected from DOTA-TOC and DOTA-TATE, more preferably is DOTA-TATE.
32. The method any of the embodiments 26-31, wherein the radiopharmaceutical compound is [¹⁷⁷Lu] Lu-DOTA-TOC (¹⁷⁷Lu-edotreotide) or [¹⁷⁷Lu] Lu-DOTA-TATE (¹⁷⁷Lu-oxodotreotide), more preferably [¹⁷⁷Lu] Lu-DOTA-TATE (¹⁷⁷Lu-oxodotreotide).
33. The method of any of the embodiments 26-32, wherein said subject has not received prior systematic treatment for SCLC, in particular ES-SCLC, in particular said subject has not received prior chemotherapy for treating SCLC, in particular ES-SCLC.
34. The method of any of the embodiments 26-33, wherein said subject is newly diagnosed with SCLC, in particular ES-SCLC.
35. The method of the embodiments 26-34, wherein said subject has been selected for the treatment by SPECT/CT or PET/CT or SPECT/MRI, PET/MRI imaging with the same organic compound as used for the radiopharmaceutical treatment but with a radiometal suitable for imaging, preferably 68Ga, 67Ga or 64Cu, more preferably 68Ga.
36. The method of any of the embodiments 26-35, wherein said subject has been diagnosed as SSTR positive by imaging positron emission tomography (PET) scan in at least one target or non-target lesion, preferably with [68Ga] Ga-DOTA-TATE imaging PET scan.
37. The method of any of the embodiments 26-36, wherein said one or more chemotherapeutic agents include carboplatin and etoposide.
38. The method of any of the embodiments 26-37, wherein said radiopharmaceutical compound is administered 1 to 8 times, preferably 2 to 7 times, for example 4 to 6 times, wherein there is a treatment interval between every two administrations of said radiopharmaceutical compound.
39. The method of any of the embodiments 26-38, wherein each administration of said radiopharmaceutical compound comprises a treatment interval of 2 weeks, or 3 weeks, or 4 weeks, or 5 weeks or even 6 weeks, preferably 3 and/or 6 weeks.
40. The method of any of the embodiments 26-39, wherein said one or more chemotherapeutic agents is (are) administered in combination, preferably concomitantly during an induction period comprising two administrations of said radiopharmaceutical compound, preferably a first administration of said radiopharmaceutical compound at week 1 of the first administration of a chemotherapeutic agent, for example on either Day 3, 4 or 5 of Week 1, and a second administration of said radiopharmaceutical compound between Week 6 and Week 8, preferably Week 7.
41. The method of embodiment 40, further comprising a maintenance period following the induction period, comprising 1 to 4 administrations of said radiopharmaceutical compound, preferably every 3 weeks.
42. The method of any of embodiments 26-41, comprising in combination, preferably concomitantly administering a therapeutically efficient amount of one or more immune-oncology (I-O) therapeutic agents, preferably selected from the group consisting of PD-1 inhibitors, PD-L1 inhibitors, CTLA4 inhibitors, LAG-3 inhibitors, TIM-3 inhibitors, TIGIT inhibitors, GITR antagonists, TGF-b inhibitors, IL15/IL15RA complexes, CD40/CD40L complexes, OX40 inhibitors, 4-1BB/CD137 complexes, ICOS inhibitors, CD47 inhibitors, VISTA inhibitors, GD-2 inhibitors, B7/H3 inhibitors, cytokines (e.g., interferon, interlukin), cellular immunotherapies, and cancer vaccines, more preferably are PD-1 inhibitors, PD-L1 inhibitors, CTLA4 inhibitors, or a combination thereof. In some embodiments, the inhibitors used herein are antibodies.
43. The method of embodiment 42, wherein
   (i) said one or more chemotherapeutic agents is (are) administered in combination, preferably concomitantly during an induction period comprising two administrations of said radiopharmaceutical compound, preferably a first administration of said radiopharmaceutical compound at Week 1 of the first administration of a chemotherapeutic agent, for example on either Day 3, 4 or 5 of Week 1, and a second administration of said radiopharmaceutical compound between Week 6 and Week 8, preferably Week 7, and
   (ii) said immune-oncology agent is administered in combination, preferably concomitantly with said chemotherapeutic agent on Week 1, preferably the day of first administration of a chemotherapeutic agent and every 3 weeks during the induction period.
44. The method of embodiment 43, further comprising a maintenance period following the induction period comprising
   (iii) 1 to 4 administrations of said radiopharmaceutical compound every 3 weeks and
   (iv) 1 to 4 administration of said immune-oncology agent every 3 weeks.
45. The method of any of embodiments 42 to 44, wherein said PD-1, PD-L1 or CTLA-4 inhibitors are selected from the group consisting of anti-PD1, anti-PD-L1 or anti-CTLA-4 antibodies, for example selected from the group consisting of nivolumab (Bristol-Myers Squibb), ipilimumab, PDR001/spartalizumab (Novartis), Keytruda/pembrolizumab/MK-3475/lambrolizumab (Merk & Co), pidilizumab, durvalumab/MEDI4736, atezolizumab/MPDL3280A/Tecentriq/RG7446 (Roche), avelumab, MEDI0680 (AMP-514, Medimmune), REGN2810/Cemiplimab (Regeneron), TSR-042/Dostarlimab/Dostarlimab-gxly (Tesaro), PF-06801591/Sananlimab (Pfizer), BGB-A317/tislelizumab (Beigene), BGB-108, INCSHR1210/Camrelizumab (Incyte), and AMP-224 (Amplimmune).
46. The method of embodiment 45, wherein said PD-1, PD-L1 or CTLA-4 inhibitor is tislelizumab, and is preferably administered at a dose of from about 200 mg to about 500 mg, more preferably from about 200 mg to about 400 mg, even more preferably either from about 200 mg to about 300 mg, even more preferably either about 200 mg or about 300 mg, even more preferably 200 mg.
47. The method of any of embodiments 26-46 wherein said subject has not received prior systematic treatment for SCLC, in particular ES-SCLC, in particular said subject has not received prior chemotherapy for treating SCLC, in particular ES-SCLC, wherein said radiopharmaceutic compound is [¹⁷⁷Lu] Lu-DOTA-TATE, said one or more chemotherapeutic agent is carboplatin (preferably carboplatin AUC 5 on day 1 of every 3-week-cycle) and etoposide (preferably administered at a daily dose of 100 mg/m2 on days 1, 2 and 3 of every 3-week-cycle), and said use further comprises administering a therapeutically efficient amount of tislelizumab in combination, preferably concomitantly with [177LU] Lu-DOTA-TATE administration.
48. The method of embodiment 47, wherein
   (i) said carboplatin and etoposide are administered in combination, preferably concomitantly during an induction period comprising two administrations of [177LU] Lu-DOTA-TATE, preferably a first administration of [177LU] Lu-DOTA-TATE at Week 1 after the first administration of carboplatin and/or etoposide, for example on either Day 3, 4 or 5 of Week 1, and a second administration of [177LU] Lu-DOTA-TATE between Week 6 and Week 8, preferably Week 7, and
   (ii) tislelizumab is administered in combination, preferably concomitantly with carboplatin and etoposide on Week 1, preferably the day of first administration of carboplatin and every 3 weeks during the induction period.
49. The method of embodiment 48, which further comprises a maintenance period, following the induction period, comprising
   (iii) 1 to 4 administrations of [177LU] Lu-DOTA-TATE every 3 weeks and
   (iv) 1 to 4 administrations of tislelizumab agent every 3 weeks.
50. The method of any of embodiments 26-49, wherein said radiopharmaceutical compound is administered at a dose (i.e. daily dose, dose for each administration, non-cumulative dose) ranging between 0.925 GBq (25 mCito 29.6 GBq (800 mCi), preferably between 1.48 GBq (40 mCi) to 18.5 GBq (500 mCi), preferably between 1.85 GBq (50 mCi) to 14.8 GBq (400 mCi), more preferably between 3.7 GBq (100 mCi) to 11.1 GBq (300 mCi), even more preferably of around 3.7 GBq (100 mCi), 5.55 GBq (150 mCi), 7.4 GBq (200 mCi) or 9.25 GBq (250 mCi).
51. Use of a radiopharmaceutical compound in the manufacture of a drug for treating small cell lung cancer (SCLC), in particular extensive stage small cell lung cancer (ES-SCLC), in a human subject in need thereof, wherein a therapeutically efficient amount of radiopharmaceutical compound comprising a somatostatin receptor binding molecule is administered to said subject, in combination, preferably concomitantly, with a therapeutically efficient amount of one or more chemotherapeutic agents.
52. Use of embodiment 51, wherein said radiopharmaceutical compound is a compound of formula:
   M-C—S—P wherein:
   M is a radionuclide;
   C is a chelating agent capable of chelating said radionuclide;
   S is an optional spacer covalently linked between C and P;
   P is a somatostatin receptor binding peptide covalently linked to C, either directly or indirectly via S.
53. Use of embodiment 52, wherein M is selected from ⁹⁰Y, ¹³¹I, ¹²¹Sn, ¹⁸⁶Re, ¹⁸⁸Re, ⁶⁴Cu, ⁶⁷Cu, ⁵⁹Fe, ⁸⁹Sr, ¹⁹⁸Au, ²⁰³Hg, ²¹²Pb, ¹⁶⁵Dy, ¹⁰³Ru, ¹⁴⁹Tb, ¹⁶¹Tb, ²¹³Bi, ¹⁶⁶Ho, ¹⁶⁵Er, ¹⁶⁹Er, ¹⁵³Sm, 177Lu, ²¹³Bi, ²²³Ra, ²²⁵ Ac, ²²⁷ Ac, ²²⁷Th, ²¹¹At, ⁶⁷Cu, ¹⁸⁶Re, ¹⁸⁸Re, ¹⁶¹Tb, ¹⁷⁵Yb, ¹⁰⁵Rh, ¹⁶⁶Dy, ¹⁹⁹ Au, ⁴⁴Sc, ¹⁴⁹Pm, ¹⁵¹Pm, ¹⁴²Pr, ¹⁴³Pr, ⁷⁶As, ¹¹¹Ag and ⁴⁷Sc, preferably is ¹⁷⁷Lu.
54. Use according to embodiment 52 or 53, wherein C is selected from DOTA (tetraxetan), trizoxetan, DOTAGA, DTPA, NTA, EDTA, DO3A, TETA, NOTA, NOTAGA, NODAGA, NODAPA, and AAZTA (e.g. AAZTA5) chelating agent, preferably is DOTA, DOTAGA, NOTA or DTPA chelating agent, and more preferably is DOTA chelating agent.
55. Use according to any of the embodiments 51-54, wherein P is selected from octreotide, octreotate, satoreotide, lanreotide, vapreotide, and pasireotide, preferably selected from octreotide and octreotate.
56. Use according to any of the embodiments 51-55, wherein the radiopharmaceutical compound is selected from DOTA-OC, DOTA-TOC (edotreotide), DOTA-NOC, DOTA-TATE (oxodotreotide), satoreotide tetraxetan, DOTA-LAN, and DOTA-VAP, preferably selected from DOTA-TOC and DOTA-TATE, more preferably is DOTA-TATE.
57. Use according to any of the embodiments 51-56, wherein the radiopharmaceutical compound is [¹⁷⁷Lu] Lu-DOTA-TOC (¹⁷⁷Lu-edotreotide) or [¹⁷⁷Lu] Lu-DOTA-TATE (¹⁷⁷Lu-oxodotreotide), more preferably [¹⁷⁷Lu] Lu-DOTA-TATE (¹⁷⁷Lu-oxodotreotide).
58. Use according to any of the embodiments 51-57, wherein said subject has not received prior systematic treatment for SCLC, in particular ES-SCLC, in particular said subject has not received prior chemotherapy for treating SCLC, in particular ES-SCLC.
59. Use according to any of the embodiments 51-58, wherein said subject is newly diagnosed with SCLC, in particular ES-SCLC.
60. Use according to any of the embodiments 51-59, wherein said subject has been selected for the treatment by SPECT/CT or PET/CT or SPECT/MRI, PET/MRI imaging with the same organic compound as used for the radiopharmaceutical treatment but with a radiometal suitable for imaging, preferably 68Ga, 67Ga or 64Cu, more preferably 68Ga.
61. Use according to any of the embodiments 51-60, wherein said subject has been diagnosed as SSTR positive by imaging positron emission tomography (PET) scan in at least one target or non-target lesion, preferably with [68Ga] Ga-DOTA-TATE imaging PET scan.
62. Use according to any of the embodiments 51-61, wherein said one or more chemotherapeutic agents include carboplatin (preferably carboplatin AUC 5 on day 1 of every 3-week-cycle) and etoposide (preferably administered at a daily dose of 100 mg/m2 on days 1, 2 and 3 of every 3-week-cycle).
63. Use according to any of the embodiments 51-62, wherein said radiopharmaceutical compound is administered 1 to 8 times, preferably 2 to 7 times, for example 4 to 6 times, wherein there is a treatment interval between every two administrations of said radiopharmaceutical compound.
64. Use according to any of the embodiments 51-63, wherein each administration of said radiopharmaceutical compound comprises a treatment interval of 2 weeks, or 3 weeks, or 4 weeks, or 5 weeks or even 6 weeks, preferably 3 and/or 6 weeks.
65. Use according to any of the embodiments 51-64, wherein said one or more chemotherapeutic agents is (are) administered in combination, preferably concomitantly during an induction period comprising two administrations of said radiopharmaceutical compound, preferably a first administration of said radiopharmaceutical compound at week 1 of the first administration of a chemotherapeutic agent, for example on either Day 3, 4 or 5 of Week 1, and a second administration of said radiopharmaceutical compound between Week 6 and Week 8, preferably Week 7.
66. Use according to embodiment 65, further comprising a maintenance period following the induction period, comprising 1 to 4 administrations of said radiopharmaceutical compound, preferably every 3 weeks.
67. Use according to any of embodiments 51-66, comprising in combination, preferably concomitantly administering a therapeutically efficient amount of one or two more immune-oncology (I-O) therapeutic agents, preferably selected from the group consisting of PD-1 inhibitors, PD-L1 inhibitors, or CTLA4 inhibitors, LAG-3 inhibitors, TIM-3 inhibitors, TIGIT inhibitors, GITR antagonists, TGF-b inhibitors, IL15/IL15RA complexes, CD40/CD40L complexes, OX40 inhibitors, 4-1BB/CD137 complexes, ICOS inhibitors, CD47 inhibitors, VISTA inhibitors, GD-2 inhibitors, and B7/H3 inhibitors, cytokines (e.g., interferon, interlukin), cellular immunotherapies, and cancer vaccines, more preferably are PD-1 inhibitors, PD-L1 inhibitors, or CTLA4 inhibitors, or a combination thereof. In some embodiments, the inhibitors used herein are antibodies.
68. Use according to embodiment 67, wherein
   (iii) said one or more chemotherapeutic agents is (are) administered in combination, preferably concomitantly during an induction period comprising two administrations of said radiopharmaceutical compound, preferably a first administration of said radiopharmaceutical compound at Week 1 of the first administration of a chemotherapeutic agent, for example on either Day 3, 4 or 5 of Week 1, and a second administration of said radiopharmaceutical compound between Week 6 and Week 8, preferably Week 7, and
   (iv) said immune-oncology agent is administered in combination, preferably concomitantly with said chemotherapeutic agent on Week 1, preferably the day of first administration of a chemotherapeutic agent and every 3 weeks during the induction period.
69. Use according to embodiment 68, further comprises a maintenance period following the induction period comprising
   (v) 1 to 4 administrations of said radiopharmaceutical compound every 3 weeks and
   (vi) 1 to 4 administration of said immune-oncology agent every 3 weeks.
70. Use according to any of embodiments 67 to 69, wherein said PD-1, PD-L1 or CTLA-4 inhibitors are selected from the group consisting of anti-PD1, anti-PD-L1 or anti-CTLA-4 antibodies, for example selected from the group consisting of nivolumab (Bristol-Myers Squibb), ipilimumab, PDR001/spartalizumab (Novartis), Keytruda/pembrolizumab/MK-3475/lambrolizumab (Merk & Co), pidilizumab, durvalumab/MEDI4736, atezolizumab/MPDL3280A/Tecentriq/RG7446 (Roche), avelumab, MEDI0680 (AMP-514, Medimmune), REGN2810/Cemiplimab (Regeneron), TSR-042/Dostarlimab/Dostarlimab-gxly (Tesaro), PF-06801591/Sananlimab (Pfizer), BGB-A317/tislelizumab (Beigene), BGB-108, INCSHR1210/Camrelizumab (Incyte), and AMP-224 (Amplimmune).
71. Use according to embodiment 70, wherein said PD-1, PD-L1 or CTLA-4 inhibitors is tislelizumab, and is preferably administered at a dose of from about 200 mg to about 500 mg, more preferably from about 200 mg to about 400 mg, even more preferably either from about 200 mg to about 300 mg, even more preferably either about 200 mg or about 300 mg, even more preferably 200 mg.
72. Use according to any of embodiments 51-71 wherein said subject has not received prior systematic treatment for SCLC, in particular ES-SCLC, in particular said subject has not received prior chemotherapy for treating SCLC, in particular ES-SCLC, wherein said radiopharmaceutic compound is [¹⁷⁷Lu] Lu-DOTA-TATE, said one or more chemotherapeutic agent is carboplatin and etoposide, and said use further comprises administering a therapeutically efficient amount of tislelizumab in combination, preferably concomitantly with [177LU] Lu-DOTA-TATE administration.
73. Use according to embodiment 72, wherein
   (iii) said carboplatin and etoposide are administered in combination, preferably concomitantly during an induction period comprising two administrations of [177LU] Lu-DOTA-TATE, preferably a first administration of [177LU] Lu-DOTA-TATE at Week 1 after the first administration of carboplatin and/or etoposide, for example on either Day 3, 4 or 5 of Week 1, and a second administration of [177LU] Lu-DOTA-TATE between Week 6 and Week 8, preferably Week 7, and
   (iv) tislelizumab is administered in combination, preferably concomitantly with carboplatin and etoposide on Week 1, preferably the day of first administration of carboplatin and every 3 weeks during the induction period.
74. Use according to embodiment 73, wherein said use further comprises a maintenance period, following the induction period, comprising
   (v) 1 to 4 administrations of [177LU] Lu-DOTA-TATE every 3 weeks and
   (vi) 1 to 4 administrations of tislelizumab agent every 3 weeks.
75. Use according to any of embodiments 51-74, wherein said radiopharmaceutical compound is administered at a dose (i.e. daily dose, dose for each administration, non-cumulative dose) ranging between 0.925 GBq (25 mCito 29.6 GBq (800 mCi), preferably between 1.48 GBq (40 mCi) to 18.5 GBq (500 mCi), preferably between 1.85 GBq (50 mCi) to 14.8 GBq (400 mCi), more preferably between 3.7 GBq (100 mCi) to 11.1 GBq (300 mCi), even more preferably of around 3.7 GBq (100 mCi), 5.55 GBq (150 mCi), 7.4 GBq (200 mCi) or 9.25 GBq (250 mCi).

### DETAILED DESCRIPTION

The present disclosure relates to a method for treating small cell lung cancer (SCLC), in particular extensive stage small cell lung cancer (ES-SCLC) in a human subject in need thereof, wherein a therapeutically efficient amount of radiopharmaceutical compound comprising a somatostatin receptor binding molecule is administered to said subject, in combination, preferably concomitantly, with a therapeutically efficient amount of one or more chemotherapeutic agents.

### General Definitions

The use of the articles “a”, “an”, and “the” in both the description and claims are to be construed to cover both the singular and the plural, unless otherwise indicated herein or clearly contradicted by context. The terms “comprising”, “having”, “being of” as in e.g., a complex “of a radionuclide and a cell receptor binding organic moiety linked to a chelating agent”, “including”, and “containing” are to be construed as open terms (i.e., meaning “including but not limited to”) unless otherwise noted. Additionally, whenever “comprising” or another open-ended term is used in an embodiment, it is to be understood that the same embodiment can be more narrowly claimed using the intermediate term “consisting essentially of” or the closed term “consisting of”.

The term “about” or “ca.” has herein the meaning that the following value may vary for ±20%, preferably ±10%, more preferably ±5%, even more preferably ±2%, even more preferably ±1%.

Unless otherwise defined, “%” has herein the meaning of weight percent (wt %), also refered to as weight by weight percent (w/w %).

“total concentration” refers to the sum of one or more individual concentrations.

“aqueous solution” refers to a solution of one or more solute in water.

The phrase “treatment of” and “treating” includes the prevention, the amelioration or cessation of a disease, disorder, or a symptom thereof. In particular, with reference to the treatment of a tumor, the term “treatment” may refer to the inhibition of the growth of the tumor, or the reduction of the size of the tumor.

As used herein, “extensive stage small cell lung cancer” (also referred as “ES-SCLC”) refers to small cell lung cancer (SCLC) that has spread to other parts of the body such as the opposite lung, bone, brain, or bone marrow.

Consistent with the International System of Units, “MBq” is the abbreviation for the unit of radioactivity “megabecquerel.”

As used herein, “PET” stands for positron-emission tomography.

As used herein, “SPECT” stands for single-photon emission computed tomography.

As used herein, “MRI” stands for magnetic resonance imaging.

As used herein, “CT” stands for computed tomography.

As used herein, the terms “efficient amount” or “therapeutically efficient amount” of a compound refer to an amount of the compound that will elicit the biological or medical response of a subject, for example, ameliorate the symptoms, alleviate conditions, slow or delay disease progression, or prevent a disease.

The terms “patient” and “subject” which are used interchangeably refer to a human being, including for example a subject that has cancer.

“for commercial use” refers to the drug product, e.g. a pharmaceutical aqueous solution, is able to obtain (preferably has obtained) marketing authorization by health authorities, e.g. US-FDA or EMA, by complying with all drug product quality and stability requirements as demanded by such health authorities, is able to be manufactured (preferably is manufactured) from or at a pharmaceutical production site at commercial scale followed by a quality control testing procedure, and is able to be supplied (preferably is supplied) to remotely located end users, e.g. hospitals or patients.

“combination therapy”, “co-administration”, “combined administration” or “concomitant administration” refers to a combined administration of at least two therapeutic agents, where a first agent, typically a radiopharmaceutical compound is administered at the same time or separately within time intervals, with a second agent, in the same subject in need thereof, where these time intervals allow that the combined partners show a cooperative or synergistic effect for treating a disorder, e.g. a cancer. It is not intended to imply that the therapeutic agents must be administered at the same time and/or formulated for delivery together although these methods of delivery are within the scope described herein. The radiopharmaceutical compound can be administered concurrently with or prior to, or subsequent to one or more other additional therapies or therapeutic agents. The terms are also meant to encompass treatment regimens in which the agents are not necessarily administered by the same route of administration.

As used herein the term “radiopharmaceutical” refers to a pharmaceutical compound which is labelled with a radionuclide element, typically of metallic nature. A radiopharmaceutical compound may be used in peptide receptor radionclide therapy (PRRT).

As used herein, the term “PRRT” or “peptide receptor radionclide therapy” refers to a molecularly targeted radiation therapy involving the systemic administration of a radiolabeled peptide (e.g., ¹⁷⁷Lu-dotatate), which is designed to target receptors overexpressed on tumors (e.g., somatostatin receptor subtype 2) with high affinity and specificity.

### The Radiopharmaceutical Compound for Use in the Treatment Methods of the Disclosure

The radiopharmaceutical compound for use in the treatment methods of the present disclosure is a somatostating receptor (SSTR) binding compound which comprises a radionuclide and which has specific binding affinity to SSTR, for example at least somatostatin receptor subtype 2 (SSTR2).

In specific embodiments, said radiopharmaceutical compound for use as described herein is a compound of formula

M-C—S—P wherein:
M is a radionuclide;
C is a chelating agent capable of chelating said radionuclide;
S is an optional spacer covalently linked between C and P;
P is a somatostatin receptor binding peptide covalently linked to C, for example via its N-terminal end, either directly or indirectly via S.

Such radiopharmaceutical compound may be selected from octreotide, octreotate, lanreotide, vapreotide, and pasireotide, preferably selected from octreotide and octreotate.

In some embodiments of the disclosure, the radionuclide M is selected from radionuclide isotope suitable for nuclear medicine therapy or peptide receptor radionuclide therapy (PRRT).

Examples of such radionuclide M suitable for PRRT includes without limitation ⁹⁰Y, ¹³¹I, ¹²¹Sn, ¹⁸⁶Re, ¹⁸⁸Re, ⁶⁴Cu, ⁶⁷Cu, ⁵⁹Fe, ⁸⁹Sr, ¹⁹⁸Au, ²⁰³Hg, ²¹²Pb, ¹⁶⁵Dy, ¹⁰³Ru, ¹⁴⁹Tb, ¹⁶¹Tb, ²¹³Bi, ¹⁶⁶Ho, ¹⁶⁵Er, ¹⁶⁹Er, ¹⁵³Sm, ¹⁷⁷Lu, ²¹³Bi, ²²³Ra, ²²⁵Ac, ²²⁷Ac, ²²⁷Th, ²¹¹At, ⁶⁷Cu, ¹⁸⁶Re, ¹⁸⁸Re, ¹⁶¹Tb, ¹⁷⁵Yb, ¹⁰⁵Rh, ¹⁶⁶Dy, ¹⁹⁹Au, ⁴⁴Sc, ¹⁴⁹Pm, ¹⁵¹Pm, ¹⁴²Pr, ¹⁴³Pr, ⁷⁶As, ¹¹¹Ag and ⁴⁷Sc, preferably is ¹⁷⁷Lu.

As used herein, the term “chelating agent” refers to an organic moiety comprising functional groups that are able to form non-covalent bonds with the radionuclide and, thereby, form stable radionuclide complex. The chelating agent in the context of the present disclosure may be 1,4,7,10-Tetraazacyclododecane-1,4,7,10-tetraacetic acid (DOTA) (tetraxetan), trizoxetan, 1,4,7,10-tetraazacyclododececane, 1 (glutaric acid)-4,7,10-triacetic acid (DOTAGA), diethylentriaminepentaacetic acid (DTPA), nitrilotriacetic acid (NTA), ethylenediaminetetraacetic acid (EDTA), 1,4,7,10-tetraazacyclododecane-1,4,7-triacetic acid (DO3A), triethylenetetramine TETA, 1,4,7-triazacyclononane-1,4,7-triacetic acid (NOTA), NOTAGA, 1-(1,3-carboxypropyl)-4,7-carboxymethyl-1,4,7-triazacyclononane (NODAGA), NODASA, NODAPA, and 1,4-bis (carboxymethyl)-6-[bis (carboxymethyl)] amino-6-methylperhydro-1,4-diazepine (AAZTA, e.g. AAZTA5). In many embodiments of the disclosure, the chelating agent is DOTA.

Such chelating agents are either directly linked to the somatostatin receptor binding peptide or connected via a linker molecule, preferably it is directly linked. The linking bond(s) is (are) either covalent or non-covalent bond(s) between the cell receptor binding organic moiety (and the linker) and the chelating agent, preferably the bond(s) is (are) covalent.

As used herein, the term “somatostatin receptor binding peptide” refers to a peptidic moiety with specific binding affinity to somatostatin receptor. Such somatostatin receptor binding peptide may be selected from octreotide, octreotate, lanreotide, vapreotide, and pasireotide, preferably selected from octreotide and octreotate.

According to many embodiments of the methods of the present disclosure, the somatostatin receptor binding peptide linked to the chelating agent is selected from DOTA-OC, DOTA-TOC (edotreotide), DOTA-NOC, DOTA-TATE (oxodotreotide), satoreotide tetraxetan, DOTA-LAN, and DOTA-VAP. In many of these embodiments, the somatostatin receptor binding peptide is DOTA-TOC or DOTA-TATE. In many such embodiments, the somatostatin receptor binding peptide is DOTA-TATE.

In an embodiment, the radiopharmaceutical compound of the disclosure is [¹⁷⁷Lu] Lu-DOTA-TOC (¹⁷⁷Lu-edotreotide) or [¹⁷⁷Lu] Lu-DOTA-TATE (¹⁷⁷Lu-oxodotreotide), more preferably [¹⁷⁷Lu] Lu-DOTA-TATE (¹⁷⁷Lu-oxodotreotide).

Accordingly, the cell receptor binding moiety and the chelating agent may form together the following molecules:
DOTA-OC: [DOTA°,D-Phe1] octreotide,
DOTA-TOC: [DOTA°,D-Phe1, Tyr3] octreotide, edotreotide (INN),
represented by the following formulas:

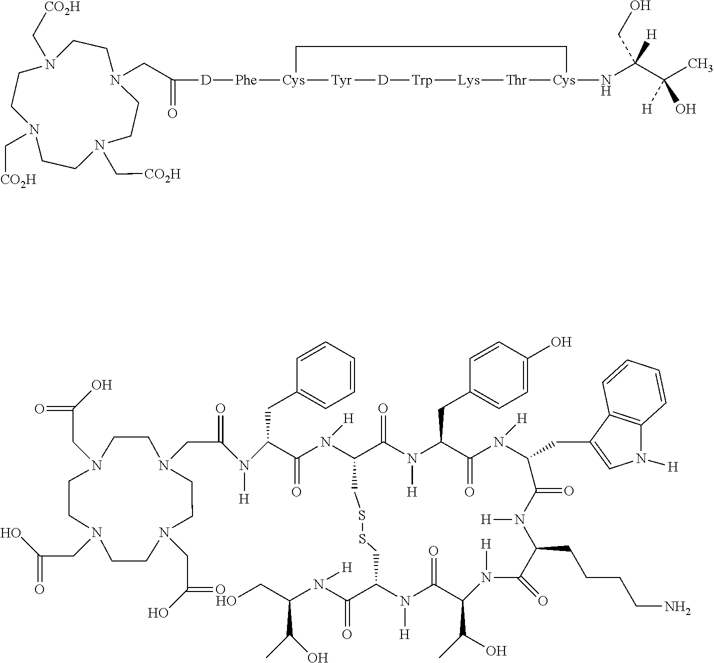

DOTA-NOC: [DOTA°, D-Phe1,1-Nal3] octreotide,
DOTA-TATE: [DOTA°,D-Phe1, Tyr3] octreotate, DOTA-Tyr3-Octreotate, DOTA-d-Phe-Cys-Tyr-d-Trp-Lys-Thr-Cys-Thr (cyclo 2,7), oxodotreotide (INN), represented by the following formula:

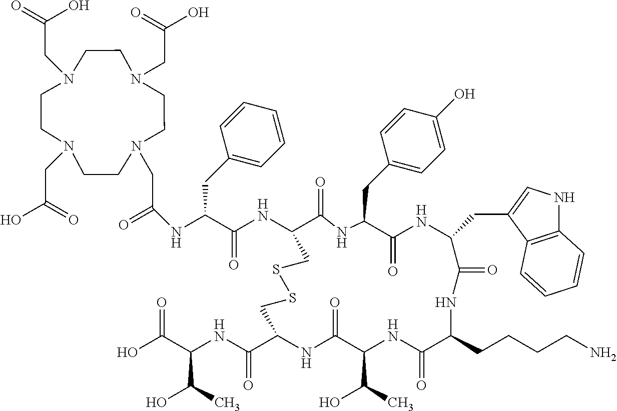 [SMILES: C[C@@H](O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

DOTA-LAN: [DOTA°,D-B-Nal1] lanreotide,
DOTA-VAP: [DOTA°,D-Phe1,Tyr] vapreotide.

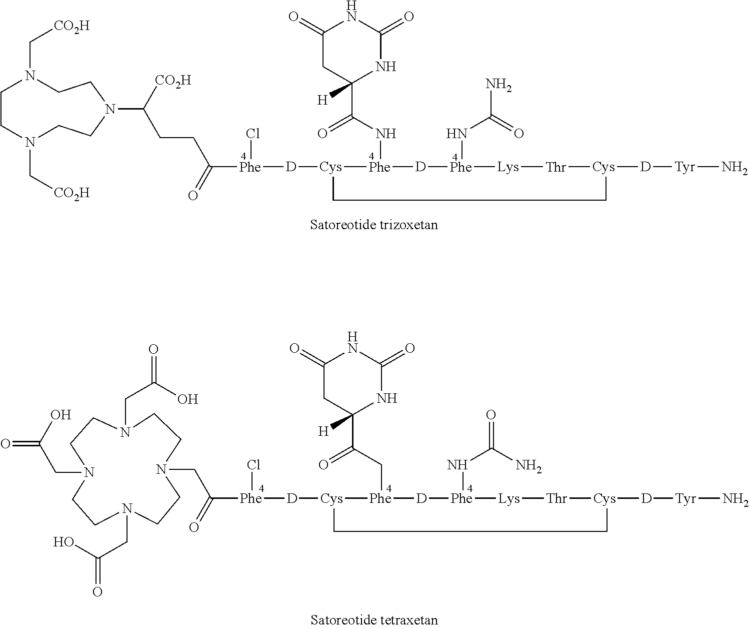

Common “cell receptor binding moiety linked to the chelating agent” molecules of the disclosure for use in the combination therapy are DOTA-TOC, DOTA-TATE, and Satoreotide tetraxetan, more preferably the molecule is DOTA-TATE.

More specifically, in many embodiments of the disclosure, the complex formed by the radionuclide and the cell receptor binding moiety linked to the chelating agent according to the present invention is [¹⁷⁷Lu] Lu-DOTA-TATE, which is also referred to as Lutetium (¹⁷⁷Lu) oxodotreotide (INN), i.e. hydrogen [N-{[4,7,10-tris (carboxylato-KO-methyl)-1,4,7,10-tetraazacyclododecan-1-yl-KAN¹, N⁴,N⁷,N¹⁰] acetyl-KO}-D-phenylalanyl-L-cysteinyl-tyrosyl-D-tryptophyl-L-lysyl-L-threonyl-L-cysteinyl-L-threoninato)] tryptophyl-L-lysyl-L-threonyl-L-cysteinyl-L-threoninato cyclic (2->7)-disulfide (4-)](¹⁷⁷Lu)lutetate(1-)
and is represented by the following formulas:

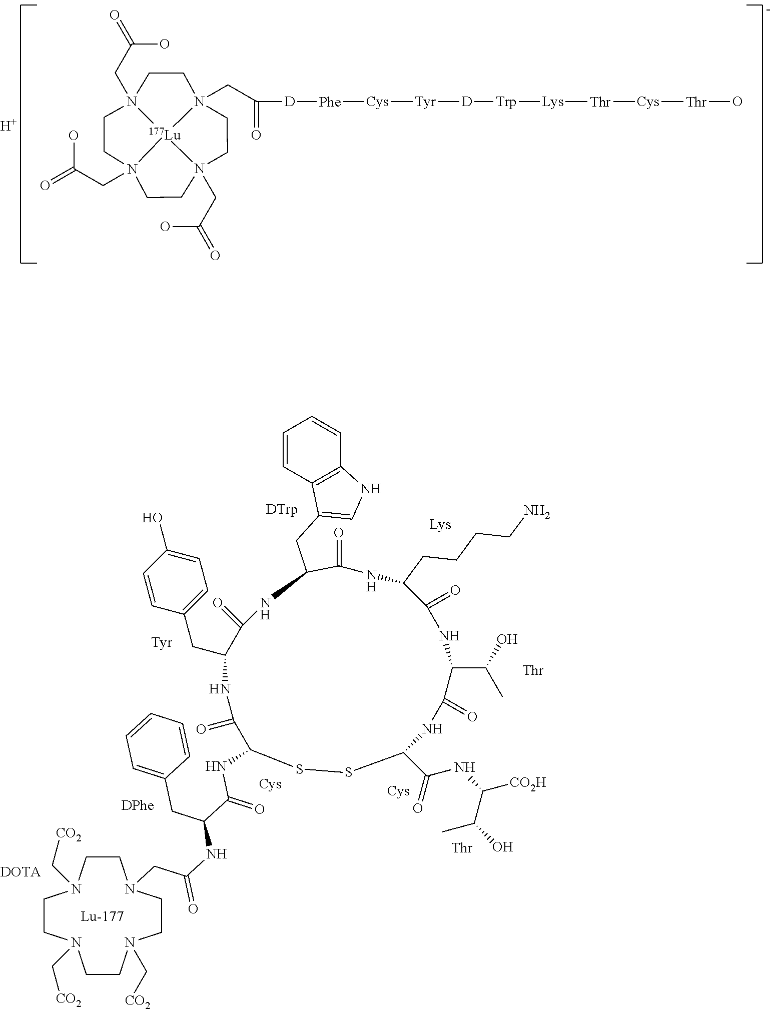

Said radiolabelled somatostatin receptor binding compound is typically formulated for administration of a therapeutically efficient amount in the subject in need thereof.

The radiolabelled somatostatin receptor binding compound can be present in a concentration providing a volumetric radioactivity of 100 MBq/mL or higher. In many embodiments of the disclosure, the volumetric radioactivity is 250 MBq/mL or higher.

In many embodiments of the disclosure, the radiolabeled somatostatin receptor binding compound can be present in a concentration providing a volumetric radioactivity comprised between 100 MBq/mL and 1000 MBq/mL, including between 250 MBq/mL and 500 MBq/mL, for example, at a concentration of about 370 MBq/mL (10 mCi/mL).

The pharmaceutically acceptable excipient can be any of those conventionally used, and is limited only by physico-chemical considerations, such as solubility and lack of reactivity with the active compound(s).

In particular, the one or more pharmaceutically acceptable excipient(s) can be selected from numerous different classes of such pharmaceutcially acceptable excipients. Examples of such classes include stabilizers against radiolytic degradation, buffers, sequestering agents and mixtures thereof.

As used herein, “stabilizer against radiolytic degradation” refers to stabilizing agent which protects organic molecules against radiolytic degradation, e.g. when a gamma ray emitted from the radionuclide is cleaving a bond between the atoms of an organic molecules and radicals are forms, those radicals are then scavenged by the stabilizer which avoids the radicals undergo any other chemical reactions which might lead to undesired, potentially ineffective or even toxic molecules. Therefore, those stabilizers are also referred to as “free radical scavengers” or in short “radical scavengers”. Other alternative terms for those stabilizers are “radiation stability enhancers”, “radiolytic stabilizers”, or simply “quenchers”.

As used herein, “sequestering agent” refers to a chelating agent suitable to complex free radionuclide metal ions in the formulation (which are not complexed with the radiolabelled peptide).

Buffers include acetate buffer, citrate buffer and phosphate buffer.

According to many embodiments of the disclosure, the pharmaceutical composition is an aqueous solution, for example an injectable formulation. According to a particular embodiment, the pharmaceutical composition is a solution for infusion.

The requirements for effective pharmaceutical carriers for injectable compositions are well-known to those of ordinary skill in the art (see, e.g., Pharmaceutics and Pharmacy Practice, J. B. Lippincott Company, Philadelphia, PA, Banker and Chalmers, eds., pages 238-250 (1982), and SHP Handbook on Injectable Drugs, Trissel, 15th ed., pages 622-630 (2009)).

The following clauses refer to various embodiments of suitable pharmaceutical aqueous solution for use in the combination methods of the present disclosure. The following clauses provided are non-limiting.
76. A pharmaceutical aqueous solution comprising
   (a) a complex formed by
      (ai) a radionuclide, and
      (aii) a cell receptor binding organic moiety linked to a chelating agent; and
   (b) at least one stabilizer against radiolytic degradation;
   wherein
   said radionuclide is present in a concentration that it provides a volumetric radioactivity of at least 100 MBq/mL, preferably of at least 250 MBq/mL.
77. The pharmaceutical aqueous solution according to embodiment 76, wherein said stabilizer(s), component (b), is (are) present in a total concentration of at least 0.2 mg/mL, preferably at least 0.5 mg/mL, more preferably at least 1.0 mg/mL, even more preferably at least 2.7 mg/mL.
78. The pharmaceutical aqueous solution according to any one of the preceding embodiments, wherein said radionuclide is present in a concentration that it provides a volumetric radioactivity of from 100 to 1000 MBq/mL, preferably from 250 to 500 MBq/mL.
79. The pharmaceutical aqueous solution according to any one of the preceding embodiments, wherein said stabilizer(s) is (are) present in a total concentration of from 0.2 to 20.0 mg/mL, preferably from 0.5 to 10.0 mg/mL, more preferably from 1.0 to 5.0 mg/mL, even more preferably from 2.7 to 4.1 mg/mL.
80. The pharmaceutical aqueous solution according to any one of the preceding embodiments, wherein the component (b) is only one stabilizers against radiolytic degradation, i.e. only a first stabilizer.
81. The pharmaceutical aqueous solution according to any one of the preceding embodiments, wherein the component (b) are at least two stabilizers against radiolytic degradation, i.e. at least a first and a second stabilizer, preferably only two stabilizers, i.e. only a first and a second stabilizer.
82. The pharmaceutical aqueous solution according to any one of the embodiments 80 to 81, wherein the first stabilizer is present in a concentration of from 0.2 to 5 mg/mL, preferably from 0.5 to 5 mg/mL, more preferably from 0.5 to 2 mg/mL, even more preferably from 0.5 to 1 mg/mL, even more preferably from 0.5 to 0.7 mg/mL.
83. The pharmaceutical aqueous solution according to embodiment 80 to 81, wherein the second stabilizer is present in a concentration of from 0.5 to 10 mg/mL, more preferably from 1.0 to 8.0 mg/mL, even more preferably from 2.0 to 5.0 mg/ml, even more preferably from 2.2 to 3.4 mg/mL.
84. The pharmaceutical aqueous solution according to any one of the preceding embodiments, wherein the stabilizer(s) is (are) selected from gentisic acid (2,5-dihydroxybenzoic acid) or salts thereof, ascorbic acid (L-ascorbic acid, vitamin C) or salts thereof (e.g. sodium ascorbate), methionine, histidine, melatonin, ethanol, and Se-methionine, preferably selected from gentisic acid or salts thereof and ascorbic acid or salts thereof.
85. The pharmaceutical aqueous solution according to any one of the preceding embodiments, which is free of ethanol.
86. The pharmaceutical aqueous solution according to any one of the embodiments 80 to 84, wherein the first stabilizer is selected from gentisic acid and ascorbic acid, preferably the first stabilizer is gentisic acid.
87. The pharmaceutical aqueous solution according to any one of the embodiments 81 to 86, wherein the second stabilizer is selected from gentisic acid and ascorbic acid, preferably the second stabilizer is ascorbic acid.
88. The pharmaceutical aqueous solution according to any one of the embodiments 81 to 82, wherein the first stabilizer is gentisic acid or a salt thereof and the second stabilizer is ascorbic acid or a salt thereof, and the ratio of the concentration (in mg/ml) of the first stabilizer to the concentration (in mg/mL) of the second stabilizer is from 1:3 to 1:7, preferably from 1:4 to 1:5.
89. The pharmaceutical aqueous solution according to any one of the preceding embodiments, wherein the radionuclide is selected from ⁹⁰Y, ¹³¹I, ¹²¹Sn, ¹⁸⁶Re, ¹⁸⁸Re, ⁶⁴Cu, ⁶⁷Cu, ⁵⁹Fe, ⁸⁹Sr, ¹⁹⁸Au, ²⁰³Hg, ²¹²Pb, ¹⁶⁵Dy, ¹⁰³Ru, ¹⁴⁹Tb, ¹⁶¹Tb, ²¹³Bi, ¹⁶⁶Ho, ¹⁶⁵Er, ¹⁶⁹Er, ¹⁵³Sm, ¹⁷⁷Lu, ²¹³Bi, ²²³Ra, ²²⁵Ac, ²²⁷Ac, ²²⁷Th, ²¹¹At, ⁶⁷Cu, ¹⁸⁶Re, ¹⁸⁸Re, ¹⁶¹Tb, ¹⁷⁵Yb, ¹⁰⁵Rh, ¹⁶⁶Dy, ¹⁹⁹Au, ⁴⁴Sc, ¹⁴⁹Pm, ¹⁵¹Pm, ¹⁴²Pr, ¹⁴³Pr, ⁷⁶As, ¹¹¹Ag and ⁴⁷Sc, preferably is ¹⁷⁷Lu.
90. The pharmaceutical aqueous solution according to any one of the preceding embodiments, wherein the cell receptor binding moiety is a somatostatin receptor binding peptide, preferably said somatostatin receptor binding peptide is selected from octreotide, octreotate, lanreotide, vapreotide and pasireotide, preferably selected from octreotide and octreotate.
91. The pharmaceutical aqueous solution according to any one of the preceding embodiments, wherein the chelating agent is selected from DOTA (tetraxetan), trizoxetan, DOTAGA, DTPA, NTA, EDTA, DO3A, TETA, NOTA, NOTAGA, NODAGA, NODASA, NODAPA, and AAZTA (e.g. AAZTA5), preferably is DOTA.
92. The pharmaceutical aqueous solution according to any one of the preceding embodiments, wherein the cell receptor binding moiety and the chelating agent form together molecules selected from DOTA-OC, DOTA-TOC (edotreotide), DOTA-NOC, DOTA-TATE (oxodotreotide), satoreotide tetraxetan, DOTA-LAN, and DOTA-VAP, preferably selected from DOTA-TOC and DOTA-TATE, more preferably is DOTA-TATE.
93. The pharmaceutical aqueous solution according to any one of the preceding embodiments, wherein the radionuclide, the cell receptor binding moiety and the chelating agent form together the complex [¹⁷⁷Lu] Lu-DOTA-TOC (¹⁷⁷Lu-edotreotide) or [¹⁷⁷Lu] Lu-DOTA-TATE (¹⁷⁷Lu-oxodotreotide), preferably [¹⁷⁷Lu] Lu-DOTA-TATE.
94. The pharmaceutical aqueous solution according to any one of the preceding embodiments, further comprising a buffer, preferably said buffer is an acetate buffer, preferably in an amount to result in a concentration of from 0.3 to 0.7 mg/ml (preferably about 0.48 mg/mL) acetic acid and from 0.4 to 0.9 mg/ml (preferably about 0.66 mg/mL) sodium acetate.
95. The pharmaceutical aqueous solution according to any one of the preceding embodiments, further comprising a sequestering agent, preferably said sequestering agent is diethylentriaminepentaacetic acid (DTPA) or a salt thereof, preferably in an amount to result in a concentration of from 0.01 to 0.10 mg/ml (preferably about 0.05 mg/mL).
96. The pharmaceutical aqueous solution according to any one of the preceding embodiments, which has a shelf life of at least 24 hours (h) at ≤25° C., at least 48 h at ≤25° C., at least 72 h at ≤25° C., of from 24 h to 120 h at ≤25° C., from 24 h to 96 h at ≤25° C., from 24 h to 84 h at ≤25° C., from 24 h to 72 h at ≤25° C., in particular has a shelf life of 72 h at ≤25° C.
97. The pharmaceutical aqueous solution according to any one of the preceding embodiments, wherein said solution is produced at commercial scale manufacturing, in particular is produced at a batch size of at least 20 GBq, at least 50 GBq, or at least 70 GBq.
98. The pharmaceutical aqueous solution according to any one of the preceding embodiments, which is ready-to-use.
99. The pharmaceutical aqueous solution according to any one of the preceding embodiments, which is for commercial use.
100. A pharmaceutical aqueous solution, comprising
   (a) a complex formed by
      (ai) the radionuclide ¹⁷⁷Lutetium (Lu-177), present in a concentration that it provides a volumetric radioactivity of from 250 to 500 MBq/mL, and
      (aii) the chelating agent linked somatostatin receptor binging organic moiety DOTA-TATE (oxodotreotide) or DOTA-TOC (edotreotide);
      (bi) gentisic acid or a salt thereof as the first stabilizer against radiolytic degradation present in a concentration of from 0.5 to 1 mg/ml;
      (bii) ascorbic acid or a salt thereof as the second stabilizer against radiolytic degradation present in a concentration of from 2.0 to 5.0 mg/mL.
101. The pharmaceutical aqueous solution according to embodiment 100, further comprising:
   (c) Diethylentriaminepentaacetic acid (DTPA) or a salt thereof in a concentration of from 0.01 to 0.10 mg/mL.
102. The pharmaceutical aqueous solution according to embodiments 100 or 101, further comprising:
   (d) acetic acid in a concentration of from 0.3 to 0.7 mg/ml and sodium acetate in a concentration from 0.4 to 0.9 mg/mL.
103. The pharmaceutical aqueous solution according to any one of the preceding embodiments wherein the stabilizer(s) is (are) present in the solution during the complex formation of components (ai) and (aii).
104. The pharmaceutical aqueous solution according to any one of embodiments 81 to 103 wherein only the first stabilizer is present during the complex formation of components (ai) and (aii), preferably in an amount to result in a concentration of from 0.5 to 5 mg/mL, more preferably from 0.5 to 2 mg/mL, even more preferably from 0.5 to 1 mg/mL, even more preferably from 0.5 to 0.7 mg/mL, in the final solution.
105. The pharmaceutical aqueous solution according to any one of embodiments 81 to 104 wherein a part of the amount of the second stabilizer is already present in the solution during the complex formation of components (ai) and (aii) and another part of the amount of the second stabilizer is added after the complex formation of components (ai) and (aii).
106. The pharmaceutical aqueous solution according to any one of embodiments 81 to 105 wherein the second stabilizer is added after the complex formation of components (ai) and (aii).
107. The pharmaceutical aqueous solution according to embodiment 81 to 106 wherein the second stabilizer is added after the complex formation of components (ai) and (aii), preferably in an amount to result in a concentration of from 0.5 to 10 mg/ml, more preferably from 1.0 to 8.0 mg/mL, even more preferably from 2.0 to 5.0 mg/mL, even more preferably from 2.2 to 3.4 mg/mL, in the final solution.
108. The pharmaceutical aqueous solution according to any one of the preceding embodiments, further comprising a sequestering agent, added after the complex formation of components (ai) and (aii), for removing any uncomplexed Lu, preferably said sequestering agent is diethylentriaminepentaacetic acid (DTPA) or a salt thereof, preferably in an amount to result in a concentration of from 0.01 to 0.10 mg/mL (preferably about 0.05 mg/mL) in the final solution.

Often, a solution for infusion of [¹⁷⁷Lu] Lu-DOTA-TATE or [¹⁷⁷Lu] Lu-DOTA-TOC such as one with specific activity concentration of 370 MBq/mL (+5%) is used in the combination methods of the present disclosure.

A particular process for manufacturing the pharmaceutical aqueous solution as defined in any one of the preceding embodiments, may comprise the process steps:
(1) Forming a complex of the radionuclide and the chelating agent linked cell receptor binding organic moiety by
   (1.1) preparing an aqueous solution comprising the radionuclide;
   (1.2) preparing an aqueous solution comprising the chelating agent linked cell receptor binding organic moiety, a first stabilizer, optionally a second stabilizer; and
   (1.3) mixing the solutions obtained in steps (1.1) and (1.2) and heating the resulting mixture;
(2) Diluting the complex solution obtained by step (1) by
   (2.1) preparing an aqueous dilution solution optionally comprising a second stabilizer; and
   (2.2.) mixing the complex solution obtained by step (1) with the dilution solution obtained by the step (2.1).

The methods of treatments disclosed herein provides combination therapy with said radiopharmaceutical compound.

More specifically, the radiopharmaceutical compound, preferably [¹⁷⁷Lu] Lu-DOTA-TATE (¹⁷⁷Lu-oxodotreotide), is used according to the present disclosure for treating SCLC, in particular ES-SCLC in a subject in need thereof wherein a therapeutically efficient amount of said radiopharmaceutical compound is administered to said subject.

In an embodiment, said radiopharmaceutical compound is administered at a dose ranging between 0.925 GBq (25 mCi) to 29.6 GBq (800 mCi), preferably between 1.48 GBq (40 mCi) to 18.5 GBq (500 mCi), preferably between 1.85 GBq (50 mCi) to 14.8 GBq (400 mCi), more preferably between 3.7 GBq (100 mCi) to 11.1 GBq (300 mCi), even more preferably of around 3.7 GBq (100 mCi), 5.55 GBq (150 mCi), 7.4 GBq (200mCi) or 9.25 GBq (250 mCi).

In another embodiment, the radiopharmaceutical compound for use is administered 1 to 8 times per treatment at the induction phase, preferably 2 to 7 times per treatment, more preferably 4 to 6 times per treatment. The administration of the radiopharmaceutical compound for use may comprises a treatment interval of 2 weeks, or 3 weeks, or 4 weeks, or 5 weeks or 6 weeks, or 7 weeks.

In specific embodiments, during an induction period wherein the radiopharmaceutical compound (preferably [¹⁷⁷LuLu]-DOTATE) is administered in combination, preferably concomitantly with the chemotherapeutic agents (preferably carboplatin and etoposide), the treatment interval for the administration of said radiopharmaceutical compound is comprised between 6 to 8 weeks, for example 7 weeks, and during the maintenance period, where the chemotherapy is stopped, the treatment interval for the administration of said radiopharmaceutical compound is reduced, for example to be comprised between 2 and 4 weeks, preferably 3 weeks.

In specific embodiments, the total (cumulative) dose administered to the subject will not exceed 55.5 (1500 mCi).

In specific embodiments, the total (cumulative) dose will exceed 800 mCi, for example comprised between 1000 and 1500 mCi.

In certain embodiments, the dose is lower during the induction period with co-administration a chemotherapeutic agent than during the maintenance phase (after the chemotherapy period), for example, each dose is comprised between 100 and 200 mCi during the induction period, and between 150 mCi and 250 mCi during the maintenance phase.

### Chemotherapy as Used in the Combination

The present disclosure provides combination therapy of PRRT with chemotherapy, for providing a synergistic anti-tumour effect, thereby treating subject with SCLC, in particular ES-SCLC.

As used herein, the term “chemotherapy” is used for the treatment of diseases of oncological nature that uses drugs to stop the growth of cancer cells, either by killing the cells or by stopping them from dividing. Chemotherapy includes without limitation alkylating agents, antimetabolites, anti-microtubule agents, topoisomerase inhibitors and cytotoxic antibiotics.

Hence, the methods of the present disclosure comprises a step of administering one or more chemotherapeutic agents in combination, preferably concomitantly with said radiopharmaceutical compound.

In preferred embodiments, said one or more chemotherapeutic agents for use in combination with the radiopharmaceutical compound as disclosed herein is selected among alkylating agents, more preferably cisplatin and derivatives, such as cisplatin or carboplatin.

The current standard of care for treating ES-SCLC patients for more than 2 decades is platinum chemotherapy (carboplatin or cisplatin) with etoposide. According, in specific embodiments, said one or more chemotherapeutic agents for use in combination with the radiopharmaceutical compound as disclosed herein is carboplatin with etoposide.

In specific embodiments, the combination therapy comprises 3-4 cycles of administration of one or more chemotherapeutic agents, e.g.; carboplatin and etoposide, for example every 3 weeks.

In specific embodiments, the combination therapy comprises 3-4 cycles of administration of carboplatin and etoposide, every 3 weeks, wherein carboplatin is administered at area under the curve (AUC) 5 and etoposide at 100 mg/m2.

In specific embodiments, the first administration of the one or more chemotherapeutic agents occurs less than 15 days, preferably less than 10 days, more preferably less than 7 days before or after the first administration of the radiopharmaceutical agent.

In specific embodiments, carboplatin is first administered at day 1, etoposide at day 1 to day 3, and the radiopharmaceutical compound (preferably [¹⁷⁷Lu] Lu-DOTA-TATE) is first administered at day 3 to day 5.

In specific embodiments, said one or more chemotherapeutic agents (typically carboplatin and etoposide) is (are) administered in combination, preferably concomitantly during an induction period comprising two administrations of said radiopharmaceutical compound (typically [¹⁷⁷Lu] Lu-DOTA-TATE), preferably a first administration of said radiopharmaceutical compound at week 1 of the first administration of the chemotherapeutic agent(s), for example on either Day 3, 4 or 5 of Week 1, and a second administration of said radiopharmaceutical compound between Week 6 and Week 8, preferably Week 7.

As used herein, “induction period” refers to the period in which said one or more chemotherapeutic agents, preferably carboplatin and etoposide, is administered to the subject wherein the period has a duration of up to 11 weeks, for example from week 1 day 1 to end of week 11 day 7.

In specific embodiments, the combination therapy comprises an induction period and a maintenance period.

As used herein, “maintenance period” refers to the period starting after the induction phase or after the period with concomitant administration of PRRT with the chemotherapy, wherein the chemotherapy is stopped while the PPRT is continued, for example starting at week 12 day 1 with a duration of up to week 25, or more.

In specific embodiments, the combination therapy comprises an induction period and a maintenance period.

wherein

during the induction period, the subject receives
(i) Four cycles of chemotherapeutic agents (for example carboplatin and etoposide), preferably carboplatin at area under the curve (AUC) 5 on day 1, and every 3 weeks and etoposide 100 mg/m2 on day 1-3, and every 3 weeks, and
(ii) Two administrations of radiopharmaceutical compound (e.g. [¹⁷⁷Lu] Lu-DOTA-TATE) during week 1 and week 7, preferably at day 3, 4 or 5 of week 1, and, during the maintenance period, the chemotherapy is stopped and the subject receives (iii) 1 to 4 administrations of said radiopharmaceutical compound (e.g. [¹⁷⁷Lu] Lu-DOTA-TATE), for example on Week 13, Week 16, Week 19 and Week 22.

### Immune-Oncology (I/O) Therapy

Recently, immune-oncology therapies have been added to the standard treatment scheme to treat SCLC patients, in combination with carboplatin-etoposide. In particular, atezolizumab is administered in combination with carboplatin-etoposide followed by atezolizumab in maintenance period (Horn L, Mansfield AS, Szczęsna A, et al (2018) N Engl J Med; 379 (23): 2220-9).

In specific embodiments, the combination therapy as disclosed in the previous sections further comprises in combination, preferably concomitantly administering a therapeutically efficient amount of one or more immune-oncology (I-O) therapeutic agents, preferably selected from the group consisting of PD-1 inhibitors, PD-L1 inhibitors, CTLA4 inhibitors, LAG-3 inhibitors, TIM-3 inhibitors, TIGIT inhibitors, GITR antagonists, TGF-b inhibitors, IL15/IL15RA complexes, CD40/CD40L complexes, OX40 inhibitors, 4-1BB/CD137 complexes, ICOS inhibitors, CD47 inhibitors, VISTA inhibitors, GD-2 inhibitors, B7/H3 inhibitors, cytokines (e.g., interferon, interlukin), cellular immunotherapies, and cancer vaccines, more preferably are PD-1 inhibitors, PD-L1 inhibitors, CTLA4 inhibitors, or a combination thereof. In some embodiments, the inhibitors used herein are antibodies.

As used herein, an immune-oncology therapeutic agents (interchangeably used with I/O agent, I/O therapy) refers to any angent or therapy that take advantage of the body's immune system to fight cancer. I/O therapies may specifically target cancer cells via the immune system, such as therapeutic cancer vaccines, CAR-T therapies, and targeted antibody therapies. I/O therapies do not always need to directly target cancer cells, they can treat cancer by enhance the ability of the immune system to attack cancer cells, such as checkpoint inhibitors and cytokines.

In specific embodiments, I/O therapeutic agents which can be used thus includes immune checkpoint inhibitors, preferably selected from the group consisting of PD-1, PD-L1, or CTLA4 inhibitors, LAG-3 inhibitors and TIM-3 inhibitors.

Examples of I/O therapeutic agents are further disclosed in WO2016/207732 and WO2020/021465 which content is incorporated herein in its entirety.

More specifically, the term “PD-1” has its general meaning in the art and refers to the programmed death-1 receptor. The term “PD-1” also refers to a type I transmembrane protein, belonging to the CD28-B7 signalling family of receptors that includes CD28, cytotoxic T-lymphocyte-associated antigen 4 (CTLA-4), inducible costimulator (ICOS), and which interacts with PD-L1.

The term “anti-PD-1 antibody” or “anti-PD-L1” has its general meaning in the art and refers to an antibody with binding affinity to PD-1 or PD-L1 respectively, and antagonist activity to PD-1, i.e., it inhibits the signal transduction cascade related to the PD-1 and inhibits PD-1 ligand binding (PD-L1; PD-L2). Such anti-PD-1 antibody or anti-PD-L1 antibody preferentially inactivates PD-1 with a greater affinity and potency, respectively, than its interaction with the other sub-types or isoforms of the CD28-B7 signalling family of receptors (CD28; CTLA-4; ICOS). Tests and assays for determining whether a compound is a PD-1 inhibitors are well known by the skilled person in the art.

Examples of said PD-1, PD-L1 or CTLA-4 inhibitors are selected from the group consisting of anti-PD1, anti-PD-L1 or anti-CTLA-4 antibodies, for example selected from the group consisting of tislelizumab, nivolumab (Bristol-Myers Squibb), ipilimumab, PDR001/spartalizumab (Novartis), Keytruda/pembrolizumab/MK-3475/lambrolizumab (Merk & Co), pidilizumab, durvalumab/MEDI4736, atezolizumab/MPDL3280A/Tecentriq/RG7446 (Roche), avelumab, MEDI0680 (AMP-514, Medimmune), REGN2810/Cemiplimab (Regeneron), TSR-042/Dostarlimab/Dostarlimab-gxly (Tesaro), PF-06801591/Sananlimab (Pfizer), BGB-A317/tislelizumab (Beigene), BGB-108, INCSHR1210/Camrelizumab (Incyte), AMP-and 224 (Amplimmune).

As used herein, the term “antibody” refers to immunoglobulin molecules and immunologically active portions of immunoglobulin molecules, i.e., molecules that contain an antigen binding site that immunospecifically binds an antigen. As such, the term “antibody” encompasses not only whole antibody molecules, but also antibody fragments as well as variants (including derivatives) of antibodies and antibody fragments.

The term “antibody” as used herein also includes bispecific or multispecific molecules. An antibody can be derivatized or linked to another functional molecule, e.g., another peptide or protein (e.g., another antibody or ligand for a receptor) to generate a bispecific molecule that binds to at least two different binding sites or target molecules. The antibody may in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results. Additionally, for the embodiment in which the bispecific molecule is multi-specific, the molecule can further include a third binding specificity, in addition to the first and second target epitope. In one embodiment, the bispecific molecules as disclosed herein comprise as a binding specificity at least one antibody, or an antibody fragment thereof, including, e.g., an Fab, Fab′, F(ab′)2, Fv, Unibody or a single chain Fv. The antibody may also be a light chain or heavy chain dimer, or any minimal fragment thereof such as a Fv or a single chain construct as described in Ladner et al. U.S. Pat. No. 4,946,778.

In natural antibodies, two heavy chains are linked to each other by disulfide bonds and each heavy chain is linked to a light chain by a disulfide bond. There are two types of light chain, lambda (A) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR).

The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences, which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. According the variable regions of the light and heavy chains typically comprise 4 framework regions and 3 CDRs of the following sequence: FR1-CDR1-FR2-CDR2-FR3-CDR3-FR4.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

An “isolated antibody”, as used herein, refers to an antibody that is substantially free of other antibodies having different antigenic specificities (e.g., an isolated antibody that specifically binds to PD-1 is substantially free of antibodies that specifically bind to other antigens than PD-1). An isolated antibody that specifically binds to PD-1 may, however, have cross-reactivity to other antigens, such as related PD-1 molecules from other species. Moreover, an isolated antibody may be substantially free of other cellular material and/or chemicals.

In preferred embodiments, said I/O agents for use in combination with the radiopharmaceutical compound as disclosed herein is selected among PD1 inhibitors, in particular anti-PD1 antibodies, anti-PD-L1 antibodies or anti-CTLA4 antibodies, more preferably BGB-A317/tislelizumab (Beigene), (Bristol-Myers nivolumab Squibb), ipilimumab, PDR001/spartalizumab (Novartis), Keytruda/pembrolizumab/MK-3475/lambrolizumab (Merk & Co), pidilizumab, durvalumab/MEDI4736, atezolizumab/MPDL3280A/Tecentriq/RG7446 (Roche), avelumab, MEDI0680 (AMP-514, Medimmune), REGN2810/Cemiplimab (Regeneron), TSR-042/Dostarlimab/Dostarlimab-gxly (Tesaro), PF-06801591/Sananlimab (Pfizer), BGB-A317/tislelizumab (Beigene), BGB-108, INCSHR1210/Camrelizumab (Incyte), and AMP-224 (Amplimmune).

In one embodiment, the PD-1 inhibitor is an anti-PD-1 antibody molecule as described in US 2015/0210769, incorporated by reference in its entirety.

In one embodiment, the anti-PD-1 antibody molecule is BGB-A317/tislelizumab as discosed in WO 2015/035606, US 2015/315274, US 2015/079109 and US 2018/111995. In one embodiment, the anti-PD-1 antibody molecule comprises one or more of the CDR sequences (or collectively all of the CDR sequences), the heavy chain or light chain variable region sequence, or the heavy chain or light chain sequence of BGB-A317/tislelizumab.

### Heavy Chain

(SEQ ID NO: 1)
QVQLQESGPGLVKPSETLSLTCTVSGFSLTSYGVHWIRQPPGKGLEWIG
VIYADGSTNYNPSLKSRVTISKDTSKNQVSLKLSSVTAADTAVYYCARA
YGNYWYIDVWGQGTTVTVSSASTKGPSVFPLAPCSRSTSESTAALGCLV
KDYFPEPVTVSWNSGALTSGVHTFPAVLQSSGLYSLSSVVTVPSSSLGT
KTYTCNVDHKPSNTKVDKRVESKYGPPCPPCPAPPVAGGPSVFLFPPKP
KDTLMISRTPEVTCVVVAVSQEDPEVQFNWYVDGVEVHNAKTKPREEQF
NSTYRVVSVLTVVHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSKLTVDKSRWQEGNVFSCSVMHEALHNHYTQKSLSL
SLGK
(SEQ ID NO: 2)
HCDR1: GFSLTSYGVH
(SEQ ID NO: 3)
HCDR2: VIYADGSTNYNPSLKS
(SEQ ID NO: 4)
HCDR3: ARAYGNYWYIDV

### Light Chain

(SEQ ID NO: 5)
DIVMTQSPDSLAVSLGERATINCKSSESVSNDVAWYQQKPGQPPKLLIN
YAFHRFTGVPDRFSGSGYGTDFTLTISSLQAEDVAVYYCHQAYSSPYTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
(SEQ ID NO: 6)
LCDR1: KSSESVSNDVA
(SEQ ID NO: 7)
LCDR2: YAFHRFT
(SEQ ID NO: 8)
LCDR3: HQAYSSPYT

In some embodiments, the PD-1 inhibitor is PDR001. PDR001 is also known as Spartalizumab. Nivolumab (clone 5C4) and other anti-PD-1 antibodies are disclosed in U.S. Pat. No. 8,008,449 and WO 2006/121168, incorporated by reference in their entirety. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety. MEDI0680 and other anti-PD-1 antibodies are disclosed in U.S. Pat. No. 9,205,148 and WO 2012/145493, incorporated by reference in their entirety. Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody molecule is nivolumab (Bristol-Myers Squibb), also known as MDX-1106, MDX-1106-04, ONO-4538, BMS-936558, or OPDIVO®. Nivolumab (clone 5C4) and other anti-PD-1 antibodies are disclosed in U.S. Pat. No. 8,008,449 and WO 2006/121168, incorporated by reference in their entirety.

In one embodiment, the anti-PD-1 antibody molecule is pembrolizumab (Merck & Co), also known as Lambrolizumab, MK-3475, MK03475, SCH-900475, or KEYTRUDA®. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety.

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is REGN2810/cemiplimab (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

In some embodiments, the PD-1 inhibitor (e.g. tislelizumab) is administered at a dose of about 200 mg to about 500 mg (e.g., about 300 mg to about 400 mg). In some embodiments, the PD-1 inhibitor is administered once every 3 weeks. In some embodiments, the PD-1 inhibitor is administered once every 4 weeks. In other embodiments, the PD-1 inhibitor is administered at a dose of about 200 mg to about 400 mg (e.g., about 300 mg) once every 3 weeks. In yet other embodiments, the PD-1 inhibitor is administered at a dose of about 300 mg to about 500 mg (e.g., about 400 mg) once every 4 weeks.

In specific embodiments, the combination therapy comprises administration of I/O therapeutic agents, for example anti-PD1 inhibitors, preferably tislelizumab, every 3-4 weeks, for example every 3 weeks.

In specific embodiments, the first administration of the I/O therapeutic agents, for example anti-PD1 inhibitors, preferably tislelizumab, occurs less than 15 days, preferably less than 10 days, more preferably less than 7 days before or after the first administration of the radiopharmaceutical agent.

In specific embodiments, the PD1 inhibitor, (preferably tislelizumab) is first administered at day 1, together with the first administration of one or more chemotherapeutic agents, and the radiopharmaceutical compound (preferably [¹⁷⁷Lu] Lu-DOTA-TATE) is first administered at day 3 to day 5.

In specific embodiments, said I/O therapeutic agents (typically anti-PD1 inhibitors, more preferably tislelizumab) is (are) administered in combination, preferably concomitantly during an induction period comprising administration of said one or more chemotherapeutic agents, and two administrations of said radiopharmaceutical compound (typically [¹⁷⁷Lu] Lu-DOTA-TATE), preferably a first administration of said radiopharmaceutical compound at week 1 of the first administration of the chemotherapeutic agent(s), for example on either Day 3, 4 or 5 of Week 1, and a second administration of said radiopharmaceutical compound between Week 6 and Week 8.

In specific embodiments of the methods of use of said radiopharmaceutical compound, said one or more chemotherapeutic agents is (are) administered in combination, preferably concomitantly during an induction period comprising two administrations of said radiopharmaceutical compound, preferably a first administration of said radiopharmaceutical compound at Week 1 of the first administration of a chemotherapeutic agent, for example on either Day 3, 4 or 5 of Week 1, and a second administration of said radiopharmaceutical compound between Week 6 and Week 8, and said immune-oncology agent is administered in combination, preferably concomitantly with said chemotherapeutic agent on Week 1, preferably the day of first administration of a chemotherapeutic agent and every 3 weeks during the induction period.

In specific embodiments, the combination therapy comprises an induction period and maintenance period wherein the maintenance period comprises
(i) 1 to 4 administrations of said radiopharmaceutical compound every 3 weeks and
(ii) 1 to 4 administration of said immune-oncology agent every 3 weeks.

In specific embodiments, the combination therapy comprises an induction period and a maintenance period.

wherein

during the induction period, the subject receives
(i) Four cycles of chemotherapeutic agents (for example carboplatin and etoposide), preferably carboplatin at area under the curve (AUC) 5 on day 1, and every 3 weeks and etoposide 100 mg/m2 on day 1-3, and every 3 weeks
(ii) Two administrations of radiopharmaceutical compound (e.g. [¹⁷⁷Lu] Lu-DOTA-TATE) during week 1 and week 7, preferably at day 3, 4 or 5 of week 1 and,
(iii) Four cycles of I/O agent (for example anti-PD1 inhibitors, typically tislelizumab),
preferably anti-PD1 inhibitors at 200 mg on dayl and every 3 weeks, and, during the maintenance period, the chemotherapy is stopped and the subject receives
(iv) 1 to 4 administrations of said radiopharmaceutical compound (e.g. [¹⁷⁷Lu] Lu-DOTA-TATE), for example on Week 13, Week 16, Week 19 and Week 22,
(v) 2 to 4 administrations of I/O agent (for example anti-PD1 inhibitors, typically tislelizumab), preferably anti-PD1 inhibitors at 200 mg every 3 weeks.

### Effect of the Combination Therapy

In certain aspects, the combined effect of the radiopharmaceutical compound (PRRT) and chemotherapy increases the overall response rate to at least 10%, 20%, 30%, 40%, or at least 50% as compared to chemotherapy alone and/or as compared to PRRT in combination with radiotherapy.

In certain aspects, the combination therapy of the present disclosure can inhibit, delay, and/or reduce tumor growth in the subject. In certain aspects, the growth of the tumor is delayed by at least 10%, 20%, 30% or 50% in comparison to an untreated control subject. In certain aspects, the growth of the tumor is delayed by at least 20% in comparison to an untreated control subject. In certain aspects, the growth of the tumor is delayed by at least 10%, 20%, 30% or 80% in comparison to the predicted growth of the tumor without the treatment. In certain aspects, the growth of the tumor is delayed by at least 20% in comparison to the predicted growth of the tumor without the treatment.

In certain aspects, the administration of the composition comprising the radiopharmaceutical composition to a subject eligible for said treatment can increase the length of survival of the subject. In certain aspects, the increase in survival is in comparison to an untreated control subject, or in comparison to a control subject treated with the standard of care for ES-SCLC subject, typically a chemotherapy of carboplatin and etoposide, optionally in combination with an I/O therapeutic agent, such as anti-PD-1, anti-PD-L1 or anti-CTLA4 antibodies. In certain aspects, the increase in survival is in comparison to the predicted length of survival of the subject without the treatment. In certain aspects, the length of survival is increased by at least 10%, 20%, or 30% the length in comparison to an untreated control subject or in comparison to a control subject treated with the standard of care for ES-SCLC subject, typically a chemotherapy of carboplatin and etoposide, optionally in combination with an I/O therapeutic agent, such as anti-PD-1, anti-PD-L1 or anti-CTLA4 antibodies. In certain aspects, the length of survival is increased by at least 20% the length in comparison to an untreated control subject. In certain aspects, the length of survival is increased by at least 10%, 20%, or 30% the length in comparison to the predicted length of survival of the subject without the treatment. In certain aspects, the length of survival is increased by at least 20% the length in comparison to the predicted length of survival of the subject without the treatment. In certain aspects, the length of survival is increased by at least one week, two weeks, one month, two months, three months, six months, one year, two years, or three years in comparison to an untreated control subject or in comparison to a control subject treated with the standard of care for SCLC, in particular ES-SCLC subject, typically a chemotherapy of carboplatin and etoposide, optionally in combination with an I/O therapeutic agent, such as anti-PD-1, anti-PD-L1 or anti-CTLA4 antibodies. In certain aspects, the length of survival is increased by at least one month, three months, or six months in comparison to an untreated control subject. In certain aspects, the length of survival is increased by at least one week, two weeks, one month, two months, three months, six months, one year, two years, or three years in comparison to the predicted length of survival of the subject without the treatment or in comparison to predicted length of survival of a control subject treated with the standard of care for SCLC, in particular ES-SCLC subject, typically a chemotherapy of carboplatin and etoposide, optionally in combination with an I/O therapeutic agent, such as anti-PD-1, anti-PD-L1 or anti-CTLA4 antibodies. In certain aspects, the length of survival is increased by at least one month, three months, or six months in comparison to the predicted length of survival of the subject without the treatment or in comparison to the predicted length of survival of a control subject treated with the standard of care for SCLC, in particular ES-SCLC subject, typically a chemotherapy of carboplatin and etoposide, optionally in combination with an I/O therapeutic agent, such as anti-PD-1, anti-PD-L1 or anti-CTLA4 antibodies.

### Methods for Selecting a Subject for the Combination Treatment

In certain embodiments of the disclosure, said small cell lung cancer is SSTR positive disease. In an embodiment, the subject is selected for the treatment by SPECT/CT or PET/CT or SPECT/MRI, PET/MRI imaging with the same organic compound as used for the PRRT but with a radiometal suitable for imaging i.e. imaging radiopharmaceutical compound. Typical radiometal suitable for use as contrast agent in imaging include the following: ¹¹¹In, ^{133m}In, ^{99m}Tc, ^{94m}Tc, ⁶⁷Ga, ⁶⁶Ga, ⁶⁸Ga, ⁵²Fe, ⁷²As, ⁹⁷Ru, ²⁰³Pb, ⁶²Cu, ⁶⁴Cu, ⁶¹Cu ¹⁷⁷Lu, ⁸⁶Y, ⁵¹Cr, ^{52m}Mn, ¹⁵⁷Gd, ¹⁶⁹Yb, ¹⁷²Tm, ^{117m}Sn, ¹²³I, ¹²⁴I, ¹²⁵I, ¹⁸F, A/¹⁸F, ¹⁵²Tb, ¹⁵⁵Tb, ⁸²Rb, ⁸⁹Zr, ⁴³Sc, ⁴⁴Sc.

According to a preferred embodiment, the radiometal suitable for imaging is 67Ga, 68Ga or 64Cu, preferably 68Ga.

In an embodiment, in particular where [¹⁷⁷Lu] Lu-DOTA-TATE is used for the PRRT in the combination therapy, the subject is selected by evaluating the 68Ga-DOTA-TATE uptake by PET/CT or PET/MRI scan at the tumor region.

Thus, the disclosure also relates to methods for determining whether a human patient having SSTR-positive SCLC, in particular ES-SCLC is eligible for said combination therapy as disclosed herein, said method comprising the steps of:
(i) administering an efficient amount of an imaging radiopharmaceutical compound as a contrast agent for imaging the uptake of said radiopharmaceutical compound,
(ii) acquiring an image by PET/MRI or PET/CT of said patient, and
(iii) comparing with a control image.

The objective of the above method is to select the patient with SSTR-positive tumors for the combination therapy, i.e. which patients with tumors which can be detected by evaluating the uptake of a imaging radiopharmaceutical SSTR binding compound, typically labelled DOTA-TATE, by PET/MRI or PET/CT imaging after injection of said imaging radiopharmaceutical compound as contrast agent.

Advantageously, a SSTR-positive patient shows statistically better response to a treatment as compared to a randomized patient population (i.e. which has not been selected by the selection step of the present method), and/or which shows less side effects to a treatment as compared to a randomized patient population (i.e. which has not been selected by the selection step of the present method).

In certain aspect, the 68Ga-DOTA-TATE is provided in a kit called NETSPOT® (Gallium Ga 68 dotatate (USAN)). This kit is for radiopharmaceutical preparation of [68Ga] Ga-DOTA-TATE approved in the United States of America (USA) (2016), Canada (2019) and Switzerland (2019) with the following indication: After radiolabeling with (68Ga), is a radioactive diagnostic agent indicated for use with PET for localization of SSTR-positive neuroendocrine tumors (NETs) (NETSPOT® PI).

In an embodiment, the selection of subject is performed between 10 to 28 days, preferably around 14 days prior to the first administration of the radiopharmaceutical compound.

In certain embodiment, said imaging radiopharmaeutical is administered at a dose between 1.5 MBq/kg (0.040 mCi/kg) and 2.5 MBq/kg (0.067 mCi/kg), preferably around 2 MBq/kg of body weight (0.054 mCi/kg), with a minimum dose of 100 MBq (2.7 mCi) and maximum dose of 200 MBq (5.4 mCi), typically by intravenous injection, preferably slow intravenous injection.

Images of subject's body are then acquired by PET/MRI or PET/CT imaging and the images are compared with a control image to identify whether the lesions identified by conventional imaging, for example by MRI, CT, SPECT or PET, are also identified by said imaging radiopharmaceutcal compound uptake, i.e. 68Ga-DOTA-TATE uptake. Typically, PET/MRI or PET/CT imaging is performed between 30 to 120 minutes, preferably between 60 to 90 minutes after the intravenous administration of said imaging radiopharmaceutical compound to the subject.

In a specific embodiment of the method, a subject is selected for the combination therapy of the disclosure fulfils the following condition: at least 10%, preferably more than 20%, preferably more than 30%, preferably more than 40%, preferably more than 50%, preferably more than 60%, preferably more than 70%, preferably more than 80% of the lesions as detected by conventional imaging in said subject, for example by MRI, CT, SPECT or PET, are also identified by the imaging radiopharmaceutical compound uptake, e.g. 68Ga-DOTA-TATE uptake, as determined by PET/MRI or PET/CT imaging in said subject.

In specific embodiment, the term “lesion” refers to measurable tumor lesions according to Modified RANO criteria as defined in Ellingson BM, Wen PY, Cloughesy TF. Modified Criteria for Radiographic Response Assessment in Glioblastoma Clinical Trials. Neurotherapeutics. 2017 April; 14 (2): 307-320. doi: 10.1007/s13311-016-0507-6. PMID: 28108885; PMCID: PMC5398984.

In certain aspect, said subject is newly diagnosed with SCLC, in particular ES-SCLC.

In certain aspect, said subject is SSTR-positive SCLC, in particular ES-SCLC.

In another embodiment, the subject has not received prior systemic treatment for SCLC, in particular ES-SCLC, in particular said subject has not received prior chemotherapy for treating SCLC, in particular ES-SCLC. In particular in specific embodiments, said subject is not confirmed relapse or refractory SCLC, in particular ES-SCLC after first line chemotherapy.

### EXAMPLES

### Example 1: Clinical study for treating ES-SCLC subjects

Provided herein is a protocol example describing a prospective phase lb Dose Finding Study Assessing Safety and Activity of [¹⁷⁷Lu] Lu-DOTA-TATE in Newly Diagnosed Extensive Stage Small Cell Lung Cancer (ES-SCLC) in combination with carboplatin, etoposide, and tislelizumab in induction, and with tislelizumab in maintenance treatment phase.

### 1. Protocol Summary

Purpose: This study aims to establish a safe and well tolerated dose of [¹⁷⁷Lu] Lu-DOTA-TATE in this setting and to evaluate preliminary activity of the combination treatment. The study will be essential to assess a new potential therapeutic option in patients with this aggressive cancer type.

The primary objective is to establish the recommended dose of [¹⁷⁷Lu] Lu-DOTA-TATE in combination with carboplatin, etoposide, and tislelizumab in induction treatment and with tislelizumab in maintenance treatment in newly diagnosed patients with ES-SCLC.

The secondary objectives are listed hereafter:
To characterize the safety and tolerability of [¹⁷⁷Lu] Lu-DOTA-TATE in combination with carboplatin, etoposide, and tislelizumab in induction treatment and with tislelizumab in maintenance treatment in newly diagnosed patients with ES-SCLC.
To assess the safety and tolerability of [68Ga] Ga-DOTA-TATE in patients with newly diagnosed ES-SCLC.
To assess the preliminary anti-tumor activity of [¹⁷⁷Lu] Lu-DOTA-TATE in newly diagnosed patients with ES-SCLC.
To characterize the pharmacokinetics (PK) and dosimetry of [¹⁷⁷Lu] Lu-DOTA-TATE in patients with newly diagnosed patients with ES-SCLC.
To characterize the pharmacokinetics and determine the immunogenicity of tislelizumab in patients with newly diagnosed ES-SCLC

### Study Design

This is a multi-center, open label, Phase Ib study of [¹⁷⁷Lu] Lu-DOTA-TATE in combination with carboplatin, etoposide, and tislelizumab during the induction treatment period and with tislelizumab in the maintenance treatment period, in participants with newly diagnosed ES-SCLC.

The study for each participant consists of a Screening period, a Treatment period that includes an Induction treatment period and a Maintenance treatment period, and a Follow-up period.

Eligible participants will be enrolled in cohorts of 3 to 6 participants to receive:
Four cycles of carboplatin area under the curve (AUC) 5 on Day 1 and etoposide 100 mg/m2 on Day 1-3, every 3 weeks at Weeks 1, 4, 7 and 10 in induction period
Tislelizumab 200 mg on Day 1 every 3 weeks in induction and maintenance period.
Two administrations of [¹⁷⁷Lu] Lu-DOTA-TATE during the induction period: on either Day 3, 4 or 5 of Week 1 and on Week 7 Day 3 period followed by 1 to 4 administrations during the maintenance period on Week 13 Day 1, Week 16 Day 1, Week 19 Day 1 and Week 22 Day 1, depending on the dose assessed.

Up to six different dose level combinations of [¹⁷⁷Lu] Lu-DOTA-TATE will be assessed in the study as follows:
Dose Level 1 (DL1): 100 mCi of [¹⁷⁷Lu] Lu-DOTA-TATE in the induction period and 100 mCi of [¹⁷⁷Lu] Lu-DOTA-TATE in the maintenance period.
Dose Level 2a (DL2a): 150 mCi of [¹⁷⁷Lu] Lu-DOTA-TATE in both the induction and the maintenance periods.
Dose Level 2b (DL2b): 150 mCi of [¹⁷⁷Lu] Lu-DOTA-TATE in the induction period and 200 mCi of [¹⁷⁷Lu] Lu-DOTA-TATE in the maintenance period.
Dose Level 3a (DL3a): 200 mCi of [¹⁷⁷Lu] Lu-DOTA-TATE in both the induction and maintenance periods.
Dose Level 3b (DL3b): 200 mCi of [¹⁷⁷Lu] Lu-DOTA-TATE in the induction period and 250 mCi of [¹⁷⁷Lu] Lu-DOTA-TATE in the maintenance period.
Dose Level 4 (DL4): 250 mCi of [¹⁷⁷Lu] Lu-DOTA-TATE in both the induction and maintenance periods.

The dose escalation part in this study will be guided by the dose limiting toxicity (DLT) rate observed within the first 6 weeks (42 days) of treatment. In addition to DLTs the totality of safety data available at the time will be assessed for each dose escalation decision. Dose escalation/de-escalation will be conducted using the Bayesian Optimal Interval Approach (BOIN).

### Rationale:

As this is the first study of [¹⁷⁷Lu] Lu-DOTA-TATE in patients with ES-SCLC, its efficacy and safety have not been established in this population yet. However, based on the mechanism of action of [¹⁷⁷Lu] Lu-DOTA-TATE targeting somatostatin receptor (SSTR) expressing tumor cells and available pre-clinical and clinical data, it is plausible that patients with ES-SCLC might benefit from the application of targeted radioligand therapy. This study will be essential to bring a new potential therapeutic option in ES-SCLC, and given the unmet medical need and limited treatment options, the benefit/risk assessment for patients with ES-SCLC in this phase Ib study is considered favorable.

| | |
|---|---|
| Key Inclusion | Participant is ≥18 years on the day of signing informed consent form |
| criteria | Histologically or cytologically confirmed ES-SCLC |
| | Presence of measurable disease (at least one target lesion) |
| | according to RECIST v1.1 assessed by conventional computed |
| | tomography (CT) scan |
| | SSTR positive [⁶⁸Ga]Ga-DOTA-TATE imaging positron emission |
| | tomography (PET) scan demonstrating uptake in at least one target |
| | or non-target lesion |
| | No prior systemic treatment for ES-SCLC |
| | Provision of tumor tissue to support exploratory biomarker analysis |
| | Life expectancy of ≥6 months |
| Key Exclusion | Participant has received prior therapy with an antibody or drug |
| criteria | against immune checkpoint pathways |
| | Active leptomeningeal disease or uncontrolled, untreated brain |
| | metastasis |
| | Any major surgical procedure requiring general anesthesia ≤28 days |
| | before Cycle 1 Day 1 |
| | History or current diagnosis of electrocardiogram (ECG) |
| | abnormalities indicating significant risk of safety for participants |
| | participating in the study |
| | Known hypersensitivity to the active substances or any of the |
| | excipients of the study drugs |
| | Concurrent participation in another therapeutic clinical study |
| Study | In this study, approximately 39 patients with extensive stage small cell |
| treatment | lung cancer will receive treatment with [¹⁷⁷Lu]Lu-DOTA-TATE (2 |
| | administrations during induction period at Weeks 1 and 7, and starting |
| | from Week 13, every 3 weeks during maintenance period for up to 4 |
| | administrations) plus tislelizumab (200 mg every 3 weeks for the duration |
| | of the study). Before beginning treatment, patients will receive imaging |
| | agent [⁶⁸Ga]Ga-DOTA-TATE. |
| | [¹⁷⁷Lu]Lu-DOTA-TATE, tislelizumab, and [⁶⁸Ga]Ga-DOTA-TATE will be |
| | provided by the Sponsor. The Sponsor will also provide the 2.5% Lysine- |
| | Arginine amino-acid solution for infusion (if it can't be compounded locally). |
| | Carboplatin and etoposide will be provided locally. |
| Efficacy | Tumor response will be assessed locally based on RECIST 1.1, including |
| assessments | following assessments: |
| | Chest and abdomen CT/magnetic resonance imaging (MRI) with |
| | contrast every 6 weeks (±7 days). Partial Response (PR) and |
| | Complete Response (CR) must be confirmed by repeat assessments |
| | performed not less than 4 weeks after the criteria for response are first |
| | met |
| | Brain CT or MRI (if lesions identified at baseline) every 6 weeks (±7 |
| | days); otherwise as clinically indicated |
| | Pelvis imaging (if lesions identified at baseline) every 6 weeks (±7 |
| | days); otherwise as clinically indicated |
| | Whole body bone scan as clinically indicated |
| | Localized bone CT, MRI, or x-ray (if any lesions identified on the whole |
| | body bone scan that are not visible on the chest and abdomen CT or |
| | MRI at baseline) every 6 weeks (±7 days) |
| | CT/MRI of other metastatic sites (if lesion identified at baseline) every |
| | 6 weeks (±7 days) |
| | PET/CT or PET/MRI imaging with [⁶⁸Ga]Ga-DOTA-TATE: |
| | [⁶⁸Ga]Ga-DOTA-TATE imaging PET scan at screening for |
| | somatostatin receptor imaging |
| | Imaging for dosimetry of [¹⁷⁷Lu]Lu-DOTA-TATE: |
| | Whole body planar imaging for dosimetry assessments at Week 7 |
| | SPECT/CT imaging for dosimetry assessments at Week 7 |
| | Survival status will be assessed every 8 weeks (or earlier if required) |
| | regardless of treatment discontinuation reason |

### 2. Detailed Study Design

The study for each participant consists of a Screening period, a Treatment period that includes an Induction treatment period and a Maintenance treatment period, and a Follow-up period.

### Screening Period

During the screening period of up to 28 days before starting SCLC treatment, participant eligibility will be determined according to the protocol's pre-defined inclusion and exclusion criteria. Imaging with [68Ga] Ga-DOTA-TATE should be performed as soon as possible during screening in order not to delay participant enrolment.

Participants who meet all eligibility criteria at screening can be enrolled in the study. The enrollment and [¹⁷⁷Lu] Lu-DOTA-TATE order must be performed immediately after all eligibility criteria are verified and the participant is confirmed to be eligible.

All participants receiving [68Ga] Ga-DOTA-TATE will be followed for safety with a dedicated phone call 2 days after [68Ga] Ga-DOTA-TATE administration to assess occurrence of AEs.

### Treatment Period

Treatment period will consist of induction period and maintenance period. Eligible participants will be enrolled in cohorts of 3 to 6 participants to receive:
Four cycles of carboplatin area under the curve (AUC) 5 on Day 1 and etoposide 100 mg/m2 on Day 1-3, every 3 weeks at Weeks 1, 4, 7 and 10 in induction period
Tislelizumab 200 mg on Day 1 every 3 weeks in induction and maintenance period.
Two administrations of [¹⁷⁷Lu] Lu-DOTA-TATE during the induction period: on either Day 3, 4 or 5 of Week 1 and on Week 7 Day 3 period followed by 1 to 4 administrations during the maintenance period on Week 13 Day 1, Week 16 Day 1, Week 19 Day 1 and Week 22 Day 1, depending on the dose assessed.

It is anticipated that a total of approximately 39 participants will be enrolled in this study. Enrollment of cohorts will not proceed until the Investigator receives written confirmation from the Sponsor indicating that the results of the previous cohort were evaluated, and that it is permissible to begin the new cohort.

Up to six different dose level combinations of [¹⁷⁷Lu] Lu-DOTA-TATE will be assessed in the study as follows:
Dose Level 1 (DL1): 100 mCi of [¹⁷⁷Lu] Lu-DOTA-TATE in the induction period and 100 mCi of [¹⁷⁷Lu] Lu-DOTA-TATE in the maintenance period.
Dose Level 2a (DL2a): 150 mCi of [¹⁷⁷Lu] Lu-DOTA-TATE in both the induction and the maintenance periods.
Dose Level 2b (DL2b): 150 mCi of [¹⁷⁷Lu] Lu-DOTA-TATE in the induction period and 200 mCi of [¹⁷⁷Lu] Lu-DOTA-TATE in the maintenance period.
Dose Level 3a (DL3a): 200 mCi of [¹⁷⁷Lu] Lu-DOTA-TATE in both the induction and maintenance periods.
Dose Level 3b (DL3b): 200 mCi of [¹⁷⁷Lu] Lu-DOTA-TATE in the induction period and 250 mCi of [¹⁷⁷Lu] Lu-DOTA-TATE in the maintenance period.
Dose Level 4 (DL4): 250 mCi of [¹⁷⁷Lu] Lu-DOTA-TATE in both the induction and maintenance periods.
For Dose Level 1, participants will receive 4 doses of [¹⁷⁷Lu] Lu-DOTA-TATE during the maintenance period (i.e. 6 doses of [¹⁷⁷Lu] Lu-DOTA-TATE overall; cumulative dose 600 mCi). For higher dose levels, a check will be conducted before the administration of next planned [¹⁷⁷Lu] Lu-DOTA-TATE dose that would lead to exceeding 800 mCi cumulative dose, as follows:
For Dose Level 2a: after the 3rd [¹⁷⁷Lu] Lu-DOTA-TATE dose during the maintenance period (i.e. after the 5th [¹⁷⁷Lu] Lu-DOTA-TATE dose overall).
For Dose Levels 2b and 3a: after the 2nd [¹⁷⁷Lu] Lu-DOTA-TATE dose during the maintenance period (i.e. after the 4th [¹⁷⁷Lu] Lu-DOTA-TATE dose overall).
For Dose Levels 3b and 4: after the 1st [¹⁷⁷Lu] Lu-DOTA-TATE dose during the maintenance period (i.e. after the 3rd [¹⁷⁷Lu] Lu-DOTA-TATE dose overall)

The Investigator should determine if:
The participant shows evidence of disease stabilization or response (i.e. radiological, or assessed by clinical benefit).
Has shown good tolerance to the [¹⁷⁷Lu] Lu-DOTA-TATE treatment.

If the participant meets all of the criteria above and agrees to continue with further maintenance treatment with [¹⁷⁷Lu] Lu-DOTA-TATE, the Investigator may administer a further 1-3 cycles with a cumulative dose of [¹⁷⁷Lu] Lu-DOTA-TATE exceeding 800 mCi. A maximum of 6 cycles of radioligand therapy is allowed (see next section for rationale) corresponding to a maximum of 1500 mCi.

An infusion of 2.5% Lysine-Arginine amino acid (AA) solution will be co-administered with each [¹⁷⁷Lu] Lu-DOTA-TATE dose for renal protection according to the [¹⁷⁷Lu] Lu-DOTA-TATE prescribing information. An antiemetic should be administered for prevention of infusion-related nausea and vomiting.

In order to assess the absorbed doses in critical organs and tumoral lesions of a more frequent [¹⁷⁷Lu] Lu-DOTA-TATE administration compared to Q8W administration in approved GEP-NET indication, the first three participants of each dose level (1,2a, 3a, and 4) will undergo additional assessments for dosimetry/PK during the first week after the 2nd [¹⁷⁷Lu] Lu-DOTA-TATE dose, i.e. one time during the study treatment period for each participant. In exceptional circumstances when dosimetry cannot be performed in a particular participant after the second [¹⁷⁷Lu] Lu-DOTA-TATE dose, it should be completed as soon as feasible upon a later dose. The dosimetry analysis at the 2nd [¹⁷⁷Lu] Lu-DOTA-TATE administration will be used as supportive data for the more frequent regimen in relation to the overall assessment of the participant with regards to safety and efficacy.

Up to 6 additional participants may be enrolled at a dose level already tested to provide additional safety, tolerability and pharmacokinetic data.

Treatment period for each participant will last until disease progression confirmed per RECIST 1.1 or discontinuation for another reason. In participants receiving tislelizumab in the maintenance period, treatment beyond the initial investigator-assessed, RECIST v1.1-defined disease progression is permitted provided that the participant has investigator-assessed clinical benefit and is tolerating study drug and specific conditions are met.

Upon the last dose of study treatment, an End of Treatment visit must be performed within 28 days.

### Rationale for Study Design

Despite the initial response to first-line treatments in patients with newly diagnosed ES-SCLC, their prognosis remains poor, with overall survival of approximately 10-12 months. There is an unmet medical need for novel therapeutic options that would improve prognosis of patients with this aggressive disease.

SSTR expression has been demonstrated in SCLC by both histological and imaging methods, which provides the biological rationale for the use of radioligand therapy with [¹⁷⁷Lu] Lu-DOTA-TATE in patients with this tumor type (Reisinger I, Bohuslavitzki KH, Brenner W, et al (1998) J Nucl Med; 39 (2): 224-7, Reubi JC, Waser B, Schaer JC, et al (2001) Eur J Nucl Med; 28 (7): 836-46, Lehman JM, Hoeksema MD, Staub J, et al (2019) Int J Cancer; 144:1104-14). On the basis of [¹⁷⁷Lu] Lu-DOTA-TATE efficacy in other SSTR-positive tumors, this study will evaluate the combination of [¹⁷⁷Lu] Lu-DOTA-TATE with established first line treatment in ES-SCLC, which should result in synergistic antitumor response in SSTR-positive ES-SCLC.

This is the first clinical study that will assess [¹⁷⁷Lu] Lu-DOTA-TATE in combination with carboplatin, etoposide, and tislelizumab in newly diagnosed patients with ES-SCLC. The primary objective is to evaluate several doses of [¹⁷⁷Lu] Lu-DOTA-TATE in the induction and maintenance periods, with the aim to select the appropriate dose in this setting. For this purpose, the study will evaluate DLTs during the first cycle of [¹⁷⁷Lu] Lu-DOTA-TATE and conduct dose escalation/de-escalation guided through the Bayesian Optimal Interval Approach (BOIN) (Liu S, Yuan Y (2015) Bayesian optimal interval designs for phase | clinical trials; 64 (3): 507-23; Yuan Y, Hess KR, Hilsenbeck SG, et al (2016) Clin Cancer Res; 22 (17): 4291-301).

The secondary objectives of the study were selected to evaluate overall safety of [¹⁷⁷Lu] Lu-DOTA-TATE in this new setting and to assess preliminary signs of antitumor activity of the combination. For antitumor activity assessment, progress-free survival (PFS), overall survival (OS), overall response rate (ORR), and DoR will be evaluated as these are most relevant endpoints. In addition, as [¹⁷⁷Lu] Lu-DOTA-TATE is a radioligand compound, its dosimetry and pharmacokinetics will be evaluated. The evaluation of SSTR expression will be done as part of exploratory objectives.

This study is designed in a non-randomized open-label fashion, which is considered appropriate for a phase I dose escalation study. As a standard approach for phase I trials in oncology patients, the study will be conducted at multiple centers across several countries.

In summary, this multicenter open-label clinical study will establish a safe and well-tolerated dose of [¹⁷⁷Lu] Lu-DOTA-TATE in combination with carboplatin, etoposide, and tislelizumab during the induction treatment period and with tislelizumab in the maintenance treatment period, in participants with newly diagnosed ES-SCLC. In addition, it will explore safety, preliminary antitumor activity, pharmacokinetics and dosimetry of [¹⁷⁷Lu] Lu-DOTA-TATE in this combination setting.

### Rationale for Dose/Regimen and Duration of Treatment

### Rationale for Selection of [¹⁷⁷Lu] Lu-DOTA-TATE Dose and Schedule

The approved adult regimen of [¹⁷⁷Lu] Lu-DOTA-TATE in the established GEP-NET indication consists of 4 doses (7.4 GBq/200 mCi each) administered every 8 weeks (cumulative dose: 29.6 GBq/800 mCi). It was shown that this regimen was safe and led to significant improvement of progression free survival (PFS) and quality of life in GEP-NET patients (Strosberg J, Leeuwenkamp O, Siddiqui MK (2021) Cancer Treat Rev; 93:102141; Strosberg J, Wolin E, Chasen B, et al (2018). J Clin Oncol; 36 (25): 2578-84).

In contrast to well-differentiated GEP-NET that generally has a latent course, ES-SCLC represents a highly aggressive disease with patients progressing fast even on established therapeutic regimens. In the IMPower133 study, the PFS in patients from the atezolizumab arm was 5.2 months (Horn L, Mansfield AS, Szczęsna A, et al (2018) N Engl J Med; 379 (23): 2220-9), and in the CASPIAN study, the PFS in patients from the durvalumab arm was 5.1 months (Paz-Ares L, Dvorkin M, Chen Y, et al (2019) Lancet; 394:1929-39). Due to the short PFS in this patient population, it is considered that the standard interval of 8 weeks between the [¹⁷⁷Lu] Lu-DOTA-TATE doses will not allow for delivery of cumulative radioactive dose that will be effective. Therefore, the intervals between administrations of [¹⁷⁷Lu] Lu-DOTA-TATE in this study have been shortened. Due to potential overlapping bone marrow toxicity of the combination regimen during the induction period, an every 6 weeks regimen will be evaluated, while during the maintenance period when no chemotherapy is administered the intervals between [¹⁷⁷Lu] Lu-DOTA-TATE doses will be shortened to every 3 weeks. The Q3W regimen is also synchronized with the tislelizumab treatment schedule.

As this is the first study of [¹⁷⁷Lu] Lu-DOTA-TATE in combination with carboplatin, etoposide and tislelizumab in patients with ES-SCLC, to minimize safety risks of overlapping toxicities the starting dose for the 1st cohort has been selected at 100 mCi of [¹⁷⁷Lu] Lu-DOTA-TATE in both the induction and maintenance periods. The starting dose of 100 mCi is supported by a phase II study, which assessed this dose in patients previously treated with chemotherapy, radioligand therapy and presenting risk factors or abnormal renal and bone marrow function showing favorable tolerability (Paganelli G, Sansovini M, Ambrosetti A, et al (2014) Eur J Nucl Med Mol Imaging; 41:1845-51). In this study, the 100 mCi dose (as compared to the approved dose of 200 mCi of [¹⁷⁷Lu] Lu-DOTA-TATE) reduced the absorbed radiation dose in critical organs while maintaining efficacy. In addition, 100 mCi is the dose reduction level implemented to manage toxicity with [¹⁷⁷Lu] Lu-DOTA-TATE ([¹⁷⁷Lu] Lu-DOTA-TATE Investigator Brochure).

Taking into account the favorable safety profile at 800 mCi, the possibility of exceeding the 800 mCi cumulative administered dose may be considered in patients with SCLC in view of the aggressive nature of the disease and the different prognosis in SCLC and GEP-NET patients as reflected by the median overall survival of 12.3 months in ES-SCLC (Horn L, Mansfield AS, Szczęsna A, et al (2018) N Engl J Med; 379 (23): 2220-9) vs. 48 months in GEP-NET (Strosberg J, Caplin M, Kunz P, et al (2021) Final overall survival in the phase 3 NETTER-1 study of lutetium-177-DOTATATE in patients with midgut neuroendocrine tumors [4112]. Poster presented at the American Society of Clinical Oncology Annual Meeting, held virtually on Jun. 4-8, 2021). In addition, there is a mounting body of evidence from the literature that additional doses of [¹⁷⁷Lu] Lu-DOTA-TATE can be administered if needed without additional safety signals, indicating that the cumulative dose of 800 mCi may be exceeded to maximize benefit without compromising on safety, if a careful assessment of the patient is taken into account (Strosberg J, Leeuwenkamp O, Siddiqui MK (2021) Cancer Treat Rev; 93:102141). Therefore, the number of administrations of [¹⁷⁷Lu] Lu-DOTA-TATE may vary depending on the dose level assessed, physician judgement and patient tolerability.

A population analysis was recently conducted on an adult patient population from two studies (Erasmus and NETTER-1) to identify the influential covariates on kidney and bone marrow dosimetry. The results showed a significant effect of renal function and dose on kidney and bone marrow dosimetry, which are in line with the publication of Svensson (Svensson J, Berg G, Wängberg B, et al (2015) Eur J Nucl Med Mol Imaging; 42:947-55). In the two studies, the dosimetry evaluation was performed after the first dose, and [¹⁷⁷Lu] Lu-DOTA-TATE was administered every 8 weeks on average. Given the fact that the elimination of [¹⁷⁷Lu] Lu-DOTA-TATE occurs mainly via the renal route, that the metabolization is very low (if any), and that the drug does not interact with drug transporters, its distribution and healthy tissue uptake in patients with SCLC is not expected to differ meaningfully from GEP-NET patients. In the current study, the frequency of administration increases and may influence acute toxicity, for instance time to recovery for bone marrow (BM). Therefore, dosimetry assessment is proposed after the second administration rather than after the first one to assess how a more frequent regimen correlates with dosimetry and acute toxicity (i.e. time to recovery for BM).

### Rationale for Selection of Chemotherapy and Tislelizumab Dose and Schedule

The doses and schedule of all chemotherapy drugs are based on product labelling, literature, and local guidelines.

For tislelizumab, the pharmacokinetics, safety, and efficacy data obtained from the first-in-human study BGB-A317_Study_001, as well as other clinical study data, were analyzed in aggregate to determine the recommended dose for pivotal studies. The flat dose of 200 mg intravenously once every 3 weeks was selected for further evaluation.

Rates of treatment-related adverse events and serious adverse events observed in patients receiving 2 mg/kg and 5 mg/kg once every 2 weeks and once every 3 weeks were comparable, suggesting no clear dose-dependence across these regimens. Similarly, confirmed overall response rates (ORRs) in patients treated with tislelizumab 2 mg/kg and 5 mg/kg once every 2 weeks ranged between 10% and 15%, compared to a range of 15% to 38% for patients treated at 2 mg/kg and 5 mg/kg once every 3 weeks.

According to pharmacokinetics data from BGB-A317_Study_001, Phase la, the CL of tislelizumab was found to be independent of body weight, ethnicity, and gender, and the observed serum exposure of a 200-mg dose fell between serum exposure observed after 2 mg/kg and 5 mg/kg doses (dose range with comparable safety and efficacy rates).

Additionally, no unexpected treatment-related adverse events occurred in the 200-mg fixed dose cohort (BGB-A317_Study_001, Phase la, Part 3) when compared to body-weight-based cohorts. Of the evaluable patients treated (n=13), 3 patients (23%) had a BOR of partial response (PR), 4 patients (31%) had a BOR of stable disease, and 6 patients (46%) had a BOR of progressive disease (PD). Therefore, clinical activity with a manageable and tolerable safety profile is expected to be maintained in patients receiving tislelizumab 200 mg once every 3 weeks.

Furthermore, in the phase II study of tislelizumab in combination with platinum-based chemotherapy as first-line treatment of advanced lung cancer, all patients received tislelizumab 200 mg in combination with 4-6 cycles of platinum-doublet. Encouraging antitumor activity was demonstrated, including the SCLC patient group, the drug was generally well tolerated, and distinct immune- and cell cycle-related gene signatures were associated with efficacy across cohorts (Wang Z, Zhao J, Ma Z, et al (2020) A Phase 2 Study of Tislelizumab in Combination With Platinum-Based Chemotherapy as First-line Treatment for Advanced Lung Cancer in Chinese Patients. Lung Cancer; 147:259-68).

In conclusion, based on the rationale above, the established dose of 200 mg has been selected for tislelizumab in this ES-SCLC study.

Treatment with tislelizumab in this study will continue until disease progression. However, selected participants will be allowed to continue tislelizumab treatment beyond radiographic progression per RECIST v1.1. In light of the fact that second-line therapies of ES-SCLC have an unfavorable benefit-risk profile manifested by poor efficacy and high toxicity, and the potential for pseudoprogression/tumor-immune infiltration as a result of immunotherapy, which may not be reflected without bias in the initial radiographic evaluation, participants may be considered for treatment beyond radiographic disease progression per RECIST v1.1 at the discretion of the Investigator and after discussion with the medical monitor. Participants who continue treatment beyond radiographic disease progression per RECIST v1.1 will be closely monitored clinically and tumor assessment will continue as scheduled until loss of clinical benefit.

### Rationale for Choice of Combination Drugs

In this study, [¹⁷⁷Lu] Lu-DOTA-TATE will be administered in combination with established combination of platinum-based chemotherapy and a checkpoint inhibitor. As discussed above, there is a significant unmet need in patients with ES-SCLC for new agents with novel mechanisms of action and non-overlapping toxicity, which can be combined with established treatments. Multiple targeted agents that have been investigated in the past 2 decades including those targeting tyrosine kinases (TKIs) such as epidermal growth factor receptor (EGFR) TKIs and BCR-ABL TKIs, mammalian target of rapamycin (mTOR) signaling pathway, and vascular endothelial growth factor (VEGF) signaling pathway, have been unsuccessful in showing a survival advantage in this disease (Mamdani H, Induru R, Jalal SI (2015) Transl Lung Cancer Res; 4 (5): 533-44). Platinum chemotherapy (carboplatin or cisplatin) with etoposide has remained the standard treatment in SCLC for more than 2 decades (Farago AF, Keane FK (2018) Transl Lung Cancer Res; 7 (1): 69-79).

Recently, immune checkpoint inhibitors have been added to the standard treatment scheme. The combination of atezolizumab plus carboplatin-etoposide (C/E) followed by atezolizumab in maintenance showed improved OS compared to C/E by approximately 2 months (12.3 months versus 10.3 months, HR=0.70, p=0.007) and PFS by approximately 1 month (5.2 months versus 4.3 months, HR=0.77, p=0.02) (Horn L, Mansfield AS, Szczęsna A, et al (2018) N Engl J Med; 379 (23): 2220-9). Based on this, this combination was approved for the first-line (1L) treatment of ES-SCLC by the US Food and Drug Administration (FDA) and the European Medicines Agency (EMA). Similarly, results from the randomized, phase III CASPIAN study, with median overall survival improvement from 10.3 to 13.0 months also recently led to the FDA and EMA approval of the combination of the PD-L1 inhibitor, durvalumab, plus platinum and etoposide for the 1L treatment of ES-SCLC (Paz-Ares L, Dvorkin M, Chen Y, et al (2019) Lancet; 394:1929-39).

Currently, the standard-of-care first-line treatment for patients with ES-SCLC is platinum-based chemotherapy (cisplatin or carboplatin) plus etoposide for 4 cycles in combination with checkpoint (CPI) (atezolizumab or durvalumab) inhibitors (NCCN Guidelines Small Cell Lung Cancer Version 3.2021; Dingemans AMC, Fruh M, Ardizzoni A, et al (2021) Ann Oncol; 32 (7): 839-53). However, in spite of the addition of immunotherapy in first line (1L) treatment, ES-SCLC is still an intractable disease. As SCLC is a radiosensitive tumor of neuroendocrine origin with moderate to high expression of SSTR-2 receptor target, preclinical and clinical evidence support the exploration of the targeted radioligand therapy with [¹⁷⁷Lu] Lu-DOTA-TATE in combination with carboplatin and etoposide and a checkpoint inhibitor, with the aim to improve the clinical outcome in patients with ES-SCLC in the first line clinical treatment setting.

The selection of tislelizumab as the immune check point inhibitor component in the combination scheme is based on the data from tislelizumab clinical studies, which demonstrated that tislelizumab is comparable to other checkpoint inhibitors in terms of safety and preliminary activity in patients with advanced solid tumors. In addition, it was shown that the combination of tislelizumab and various standard-of-care chemotherapies in first-line lung cancer did not lead to new safety signals compared to other checkpoint inhibitor plus chemotherapy. In a phase II study of tislelizumab in combination with platinum-based chemotherapy as first-line treatment of advanced lung cancer patients (BGB-A317-206), a cohort of 17 patients with SCLC received tislelizumab (Q3W on Day 1) plus cisplatin/carboplatin (Q3W on Day 1) and etoposide (Q3W on Days 1, 2, and 3). Encouraging antitumor activity was demonstrated, with ORR 77% median PFS 6.9 months, and median OS 15.6 months. The regimen was generally well tolerated, and distinct immune- and cell cycle-related gene signatures were associated with efficacy across cohorts (Wang Z, Zhao J, Ma Z, et al (2020) Lung Cancer; 147:259-68).

Currently tislelizumab is being investigated in a randomized, double-blind, placebo-controlled, multicenter, Phase III study (BGB-A317-312) to compare the efficacy of tislelizumab plus cisplatin or carboplatin plus etoposide and placebo plus cisplatin or carboplatin plus etoposide (Arm B) as first-line treatment in approximately 364 patients who have previously untreated ES-SCLC. Although the study has not read out yet, according to data collected from previous studies, irrespectively of monotherapy or in combination with platinum-based chemotherapy, tislelizumab has established a manageable safety profile, with the most common side effects consistent with known class effects of other anti-PD-1 antibodies.

In addition to anticancer treatment agents in this study, [68Ga] Ga-DOTA-TATE will be used as an imaging PET agent. The choice of this compound is based on the mechanism of action of [¹⁷⁷Lu] Lu-DOTA-TATE and [68Ga] Ga-DOTA-TATE, a theragnostic pair targeting SSTR receptors, providing for targeted imaging and targeted radio therapy.

### 3. Drugs for Use in the Clinical Study

TABLE 1
Investigational drugs supply

| Investigational | Pharmaceutical | Route of | Sponsor (global |
|---|---|---|---|
| Drug | Dosage Form | Administration | or local) |
| [¹⁷⁷Lu]Lu-DOTA- | Solution for infusion (7.4 | Intravenous | Sponsor (global) |
| TATE | GBq per vial) | use | |
| Tislelizumab | Solution for infusion (100 | Intravenous | Sponsor (global) |
| | mg in 10 ml isotonic | use | |
| | solution) | | |
| [⁶⁸Ga]Ga-DOTA- | Kit for radiopharmaceutical | Intravenous | Sponsor (global) |
| TATE | preparation (40 mcg per | use | |
| | kit) | | |

### [¹⁷⁷Lu] Lu-DOTA-TATE

Lutathera® is a sterile radiopharmaceutical supplied as a ready-to-use solution for infusion containing [¹⁷⁷Lu] Lu-DOTA-TATE with a volumetric activity of 370 MBq/mL at reference date and time (calibration time (tc)). The total amount of radioactivity per single dose vial is 7,400 MBq/7.4 GBq (200 mCi)+10% at the time and date of infusion.

### Tislelizumab

Tislelizumab is a monoclonal antibody formulated for intravenous injection in a single-use vial (20R glass, United States Pharmacopeia [USP] type I), containing a total of 100 mg of antibody in 10 mL of isotonic solution. Tislelizumab has been aseptically filled in a single-use glass vial with a rubber stopper and capped by an aluminum flip-off seal cap. Each vial is packaged into a single carton box.

Tislelizumab 200 mg will be administered on Day 1 of each 21-day cycle (once every 3 weeks).

### [68Ga] Ga-DOTA-TATE

NETSPOT® is a kit for radiopharmaceutical preparation of [68Ga] Ga-DOTA-TATE containing 40 mcg of DOTA-TATE. In this study, [68Ga] Ga-DOTA-TATE will be used as imaging agent to characterize SSTR during screening.

After radiolabeling with gallium (68Ga), it serves as a radioactive diagnostic agent with positron emission tomography (PET) for localization of somatostatin receptor positive tumors.

In adults, the recommended amount of radioactivity to be administered for PET imaging is 2 MBq/kg of body weight (0.054 mCi/kg), with a minimum dose of 100 MBq (2.7 mCi) and maximum dose of 200 MBq (5.4 mCi).

After reconstitution, [68Ga] Ga-DOTA-TATE will be administered by slow intravenous injection. Images can be acquired 40 to 90 minutes after the intravenous administration. All participants will be scanned during the screening period.

### Chemotherapies (Carboplatin and Etoposide)

Carboplatin and etoposide are part of the standard of care in the targeted patient population and therefore not considered as investigational agents in this study.

Participants will be treated with carboplatin AUC 5 and etoposide 100 mg/m2 intravenously on Day 1 of each 21-day cycle from Cycle 1 through Cycle 4 in the induction period. Etoposide 100 mg/m2 IV will also be administered on Days 2 and 3 in each of the 4 cycles.

Drugs should be administered sequentially on Day 1. For the timing and sequence of administration of tislelizumab and chemotherapy see Table 2.

TABLE 2
Timing of Dose Administration of Tislelizumab and Chemotherapies

| Order of | Study | Dose | | Maintenance |
|---|---|---|---|---|
| Administration | Drug | (Route) | Induction Period | Period |
| First | Tislelizumab | 200 mg | Day 1 of each 21-day cycle: | Day 1 of |
| | | (IV) | Cycle 1: Infuse over 60 | each 21-day |
| | | | minutes (wait ≥60 minutes before | cycle; Infuse |
| | | | chemotherapy at cycle 2 and | over 30 |
| | | | thereafter wait ≥30 minutes before | minutes |
| | | | chemotherapy) | |
| Second | Carboplatin | AUC 5 | Day 1 of each 21-day cycle: | N/A |
| | | (IV) | Infuse over 15-60 minutes to | |
| | | | achieve an AUC of 5 mg/ml/min | |
| | | | (Calvert formula dosing) | |
| Third | Etoposide | 100 | Day 1 to Day 3 of each 21-day | N/A |
| | | mg/m² | cycle | |
| | | (IV) | Infuse over 60 minutes | |

On days when [¹⁷⁷Lu]Lu-DOTA-TATE is administered with other study drugs, [¹⁷⁷Lu]Lu-DOTA-TATE will always be administered last.

### Additional Study Treatments

Participants will receive the 2.5% Lys-Arg amino acid solution for infusion for renal protection 5 for each [¹⁷⁷Lu] Lu-DOTA-TATE administration.

The 2.5% Lys-Arg solution must be administered intravenously with the infusion rate of 250 ml/h. The infusion should start 30 minutes prior to the start of the [¹⁷⁷Lu] Lu-DOTA-TATE infusion, and continue for a total of 4 hours (extension up to 6 hours is allowed in case of adverse reactions that require infusion interruption or slowing the infusion rate).

The composition of the 2.5% Lys-Arg solution is shown below.

TABLE 3
2.5% Lys-Arg solution composition

| Component | Quantity/1000 mL | |
|---|---|---|
| L-lysine HCL | 25 | g* |
| L-arginine HCl | 25 | g** |
| Sodium chloride 9 mg/mL (0.9%) solution for | 1 | L |
| injects or water for injection | | |

*equivalent to 20.0 g lysine
**equivalent to 20.7 g arginine

On the day of each [¹⁷⁷Lu] Lu-DOTA-TATE treatment, before the infusion with 2.5% Lys-Arg solution is started, an intravenous bolus of anti-emetic must be given with sufficient lead time as per local prescribing information. The choice of antiemetics is at the discretion of Investigator in accordance with institutional regulations (suggested options: Granisetron (3 mg), or Ondansetron (8 mg), or Tropisetron (5 mg)). Steroids should be avoided as preventive anti-emetic treatment, if possible, because of potential somatostatin receptor down-regulation.

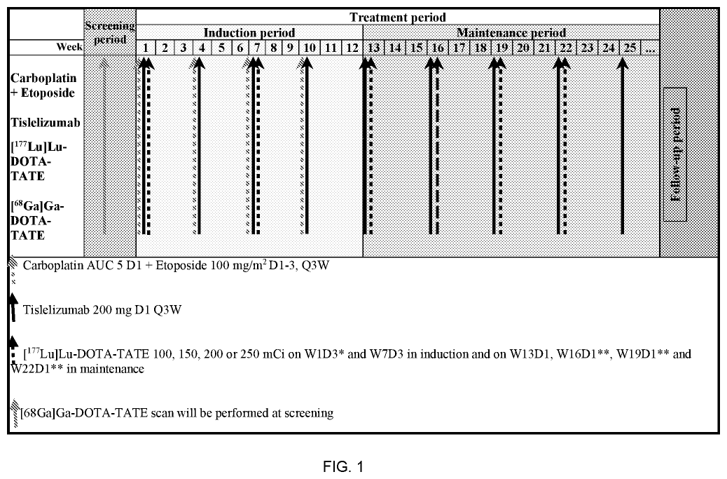

The clinical study design is represented in FIG. 1

1. A method for treating small cell lung cancer (SCLC), in particular extensive stage small cell lung cancer (ES-SCLC), in a human subject in need thereof, said method comprising administering to said subject, a therapeutically efficient amount of a radiopharmaceutical compound comprising a somatostatin receptor binding molecule, in combination, preferably concomitantly, with a therapeutically efficient amount of one or more chemotherapeutic agents.

2. The method of claim 1, wherein said radiopharmaceutical compound is a compound of formula:
M-C—S—P wherein:
M is a radionuclide;
C is a chelating agent capable of chelating said radionuclide;
S is an optional spacer covalently linked between C and P;
P is a somatostatin receptor binding peptide covalently linked to C, either directly or indirectly via S.

3. The method of claim 2, wherein M is selected from ⁹⁰Y, ¹³¹I, ¹²¹Sn, ¹⁸⁶Re, ¹⁸⁸Re, ⁶⁴Cu, ⁶⁷Cu, ⁵⁹Fe, ⁸⁹Sr, ¹⁹⁸Au, ²⁰³Hg, ²¹²Pb, ¹⁶⁵Dy, ¹⁰³Ru, ¹⁴⁹Tb, ¹⁶¹Tb, ²¹³Bi, ¹⁶⁶Ho, ¹⁶⁵Er, ¹⁶⁹Er, ¹⁵³Sm, 177Lu, ²¹³Bi, ²²³Ra, ²²⁵Ac, ²²⁷Ac, ²²⁷Th, ²¹¹At, ⁶⁷Cu, ¹⁸⁶Re, ¹⁸⁸Re, ¹⁶¹Tb, ¹⁷⁵Yb, ¹⁰⁵Rh, ¹⁶⁶ Dy, ¹⁹⁹Au, ⁴⁴Sc, ¹⁴⁹Pm, ¹⁵¹Pm, ¹⁴²Pr, ¹⁴³Pr, ⁷⁶As, ¹¹¹Ag and ⁴⁷Sc, preferably is ¹⁷⁷Lu.

4. The method of claim 2 or 3, wherein C is selected from DOTA (tetraxetan), trizoxetan, DOTAGA, DTPA, NTA, EDTA, DO3A, TETA, NOTA, NOTAGA, NODAGA, NODAPA, and AAZTA (e.g. AAZTA5) chelating agent, preferably is DOTA, DOTAGA, NOTA or DTPA chelating agent, and more preferably is DOTA chelating agent.

5. The method of any of claims 1-4, wherein P is selected from octreotide, octreotate, satoreotide, lanreotide, vapreotide, and pasireotide, preferably selected from octreotide and octreotate.

6. The method of any of claims 1-5, wherein the radiopharmaceutical compound is selected from DOTA-OC, DOTA-TOC (edotreotide), DOTA-NOC, DOTA-TATE (oxodotreotide), satoreotide tetraxetan, DOTA-LAN, and DOTA-VAP, preferably selected from DOTA-TOC and DOTA-TATE, more preferably is DOTA-TATE.

7. The method of any of claims 1-6, wherein the radiopharmaceutical compound is [¹⁷⁷Lu] Lu-DOTA-TOC (¹⁷⁷Lu-edotreotide) or [¹⁷⁷Lu] Lu-DOTA-TATE (¹⁷⁷Lu-oxodotreotide), more preferably [¹⁷⁷Lu] Lu-DOTA-TATE (¹⁷⁷Lu-oxodotreotide).

8. The method of any of claims 1-7, wherein said subject has not received prior systematic treatment for SCLC, in particular ES-SCLC, in particular said subject has not received prior chemotherapy for treating SCLC, in particular ES-SCLC.

9. The method of any of claims 1-8, wherein said subject is newly diagnosed with SCLC, in particular ES-SCLC.

10. The method of any of claims 1-9, wherein said subject has been selected for the treatment by SPECT/CT or PET/CT or SPECT/MRI, PET/MRI imaging with the same organic compound as used for the radiopharmaceutical treatment but with a radiometal suitable for imaging, preferably 68Ga, 67Ga or 64Cu, more preferably 68Ga.

11. The method of any of claims 1-10, wherein said subject has been diagnosed as SSTR positive by imaging positron emission tomography (PET) scan in at least one target or non-target lesion, preferably with [68Ga] Ga-DOTA-TATE imaging PET scan.

12. The method of any of claims 1-11, wherein said one or more chemotherapeutic agents include carboplatin and etoposide.

13. The method of any of claims 1-12, wherein said radiopharmaceutical compound is administered 1 to 8 times, preferably 2 to 7 times, for example 4 to 6 times, wherein there is a treatment interval between every two administrations of said radiopharmaceutical compound.

14. The method of any of claims 1-13, wherein each administration of said radiopharmaceutical compound comprises a treatment interval of 2 weeks, or 3 weeks, or 4 weeks, or 5 weeks or even 6 weeks, preferably 3 and/or 6 weeks.

15. The method of any of claims 1-14, wherein said one or more chemotherapeutic agents is (are) administered in combination, preferably concomitantly during an induction period comprising two administrations of said radiopharmaceutical compound, preferably a first administration of said radiopharmaceutical compound at week 1 of the first administration of a chemotherapeutic agent, for example on either Day 3, 4 or 5 of Week 1, and a second administration of said radiopharmaceutical compound between Week 6 and Week 8, preferably Week 7.

16. The method of claim 15, further comprising a maintenance period following the induction period, comprising 1 to 4 administrations of said radiopharmaceutical compound, preferably every 3 weeks.

17. The method of any of claims 1-16, comprising in combination, preferably concomitantly administering a therapeutically efficient amount of one or two more immune-oncology (I-O) therapeutic agents, preferably selected from the group consisting of PD-1 inhibitors, PD-L1 inhibitors, or CTLA4 inhibitors, LAG-3 inhibitors, TIM-3 inhibitors, TIGIT inhibitors, GITR antagonists, TGF-b inhibitors, IL15/IL15RA complexes, CD40/CD40L complexes, OX40 inhibitors, 4-1BB/CD137 complexes, ICOS inhibitors, CD47 inhibitors, VISTA inhibitors, GD-2 inhibitors, and B7/H3 inhibitors, cytokines (e.g., interferon, interlukin), cellular immunotherapies, and cancer vaccines, more preferably are PD-1 inhibitors, PD-L1 inhibitors, or CTLA4 inhibitors, or a combination thereof. In some embodiments, the inhibitors used herein are antibodies.

18. The method of claim 17, wherein
(i) said one or more chemotherapeutic agents is (are) administered in combination, preferably concomitantly during an induction period comprising two administrations of said radiopharmaceutical compound, preferably a first administration of said radiopharmaceutical compound at Week 1 of the first administration of a chemotherapeutic agent, for example on either Day 3, 4 or 5 of Week 1, and a second administration of said radiopharmaceutical compound between Week 6 and Week 8, preferably Week 7, and
(ii) said immune-oncology agent is administered in combination, preferably concomitantly with said chemotherapeutic agent on Week 1, preferably the day of first administration of a chemotherapeutic agent and every 3 weeks during the induction period.

19. The method of claim 18, further comprising a maintenance period following the induction period comprising (iii) 1 to 4 administrations of said radiopharmaceutical compound every 3 weeks and (iv) 1 to 4 administration of said immune-oncology agent every 3 weeks.

20. The method of any of claims 17-19, wherein said PD-1, PD-L1 or CTLA-4 inhibitors are selected from the group consisting of anti-PD1, anti-PD-L1 or anti-CTLA-4 antibodies, for example selected from the group consisting of nivolumab (Bristol-Myers Squibb), ipilimumab, PDR001/spartalizumab (Novartis), Keytruda/pembrolizumab/MK-3475/lambrolizumab (Merk & Co), pidilizumab, durvalumab/MEDI4736, atezolizumab/MPDL3280A/Tecentriq/RG7446 (Roche), avelumab, MEDI0680 (AMP-514, Medimmune), REGN2810/Cemiplimab (Regeneron), TSR-042/Dostarlimab/Dostarlimab-gxly (Tesaro), PF-06801591/Sananlimab (Pfizer), BGB-A317/tislelizumab (Beigene), BGB-108, INCSHR1210/Camrelizumab (Incyte), and AMP-224 (Amplimmune).

21. The method of claim 19, wherein said PD-1, PD-L1 or CTLA-4 inhibitor is tislelizumab, and is preferably administered at a dose of about 200 mg or about 300 mg.

22. The method of any of claims 1-20 wherein said subject has not received prior systematic treatment for SCLC, in particular ES-SCLC, in particular said subject has not received prior chemotherapy for treating SCLC, in particular ES-SCLC, wherein said radiopharmaceutic compound is [¹⁷⁷Lu] Lu-DOTA-TATE, said one or more chemotherapeutic agent is carboplatin (preferably carboplatin AUC 5 on day 1 of every 3-week-cycle) and etoposide (preferably administered at a daily dose of 100 mg/m2 on days 1, 2 and 3 of every 3-week-cycle)., and said use further comprises administering a therapeutically efficient amount of tislelizumab in combination, preferably concomitantly with [177LU] Lu-DOTA-TATE administration.

23. The method of embodiment 22, wherein
(i) said carboplatin and etoposide are administered in combination, preferably concomitantly during an induction period comprising two administrations of [177LU] Lu-DOTA-TATE, preferably a first administration of [177LU] Lu-DOTA-TATE at Week 1 after the first administration of carboplatin and/or etoposide, for example on either Day 3, 4 or 5 of Week 1, and a second administration of [177LU] Lu-DOTA-TATE between Week 6 and Week 8, preferably Week 7, and
(ii) tislelizumab is administered in combination, preferably concomitantly with carboplatin and etoposide on Week 1, preferably the day of first administration of carboplatin and every 3 weeks during the induction period.

24. The method of claim 23, which further comprises a maintenance period, following the induction period, comprising (iii) 1 to 4 administrations of [177LU] Lu-DOTA-TATE every 3 weeks and (iv) 1 to 4 administrations of tislelizumab agent every 3 weeks.

25. The method of any of claims 1-24, wherein said radiopharmaceutical compound is administered at a dose (i.e. daily dose, dose for each administration, non-cumulative dose) ranging between 0.925 GBq (25 mCito 29.6 GBq (800 mCi), preferably between 1.48 GBq (40 mCi) to 18.5 GBq (500 mCi), preferably between 1.85 GBq (50 mCi) to 14.8 GBq (400 mCi), more preferably between 3.7 GBq (100 mCi) to 11.1 GBq (300 mCi), even more preferably of around 3.7 GBq (100 mCi), 5.55 GBq (150 mCi), 7.4 GBq (200 mCi) or 9.25 GBq (250 mCi).


SEQUENCE LISTING (8 sequences)

SEQ ID NO: 1 - protein, 445 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEVESNGQPENNYKTTPPVLDSDGSFFLYSKLTVDKSRWQEGNVFSCSVMHEALHNHYTQKSLSLSLGK

SEQ ID NO: 2 - protein, 10 aa, Mus musculus
GFSLTSYGVH

SEQ ID NO: 3 - protein, 16 aa, synthetic construct
VIYADGSTNYNPSLKS

SEQ ID NO: 4 - protein, 12 aa, synthetic construct
ARAYGNYWYIDV

SEQ ID NO: 5 - protein, 214 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 11 aa, synthetic construct
KSSESVSNDVA

SEQ ID NO: 7 - protein, 7 aa, synthetic construct
YAFHRFT

SEQ ID NO: 8 - protein, 9 aa, synthetic construct
HQAYSSPYT